SEQUENCE LISTING (33 sequences)

SEQ ID NO: 1 - protein, 633 aa, Homo sapiens
  ref <308>: DATABASE ACCESSION NUMBER: Swissprot/Q9BZE4
  ref <309>: DATABASE ENTRY DATE: 2002-06-15
  ref <313>: RELEVANT RESIDUES: (1)..(633)
MAHYNFKKITVVPSAKDFIDLTLSKTQRKTPTVIHKHYQIHRIRHFYMRKVKFTQQNYHDRLSQILTDFPKLDDIHPFYADLMNILYDKDHYKLALGQINIAKNLVDNVAKDYVRLMKYGDSLYRCKQLKRAALGRMCTVIKRQKQSLEYLEQVRQHLSRLPTIDPNTRTLLLCGYPNVGKSSFINKVTRADVDVQPYAFTTKSLFVGHMDYKYLRWQVVDTPGILDHPLEDRNTIEMQAITALAHLRAAVLYVMDLSEQCGHGLREQLELFQNIRPLFINKPLIVVANKCDVKRIAELSEDDQKIFTDLQSEGFPVIETSTLTEEGVIKVKTEACDRLLAHRVETKMKGNKVNEVLNRLHLAIPTRRDDKERPPFIPEGVVARRKRMETEESRKKRERDLELEMGDDYILDLQKYWDLMNLSEKHDKIPEIWEGHNIADYIDPAIMKKLEELEKEEELRTAAGEYDSVSESEDEEMLEIRQLAKQIREKKKLKILESKEKNTQGPRMPRTAKKVQRTVLEKEMRSLGVDMDDKDDAHYAVQARRSRSITRKRKREDSAPPSSVARSGSCSRTPRDVSGLRDVKMVKKAKTMKNAQKKMNRLGKKGEADRHVFDMKPKHLLSGKRKAGKKDRR

SEQ ID NO: 2 - protein, 558 aa, Homo sapiens
  ref <308>: DATABASE ACCESSION NUMBER: Swissprot/O00367
  ref <309>: DATABASE ENTRY DATE: 2003-09-15
  ref <313>: RELEVANT RESIDUES: (1)..(558)
MYRYLGEALLLSRAGPAALGSASADSAALLGWARGQPAAAPQPGLALAARRHYSEAVADREDDPNFFKMVEGFFDRGASIVEDKLVEDLRTRESEEQKRNRVRGILRIIKPCNHVLSLSFPIRRDDGSWEVIEGYRAQHSQHRTPCKGGIRYSTDVSVDEVKALASLMTYKCAVVDVPFGGAKAGVKINPKNYTDNELEKITRRFTMELAKKGFIGPGIDVPAPDMSTGEREMSWIADTYASTIGHYDINAHACVTGKPISQGGIHGRISATGRGVFHGIENFINEASYMSILGMTPGFGDKTFVVQGFGNVGLHSMRYLHRFGAKCIAVGESDGSIWNPDGIDPKELEDFKLQHGSILGFPKAKPYEGSILEADCDILIPAASEKQLTKSNAPRVKAKIIAEGANGPTTPEADKIFLERNIMVIPDLYLNAGGVTVSYFEWLKNLNHVSYGRLTFKYERDSNYHLLMSVQESLERKFGKHGGTIPIVPTAEFQDRISGASEKDIVHSGLAYTMERSARQIMRTAMKYNLGLDLRTAAYVNAIEKVFKVYNEAGVTFT

SEQ ID NO: 3 - protein, 434 aa, Homo sapiens
  ref <308>: DATABASE ACCESSION NUMBER: Swiisprot/P06733
  ref <309>: DATABASE ENTRY DATE: 2003-09-15
  ref <313>: RELEVANT RESIDUES: (1)..(434)
MSILKIHAREIFDSRGNPTVEVDLFTSKGLFRAAVPSGASTGIYEALELRDNDKTRYMGKGVSKAVEHINKTIAPALVSKKLNVTEQEKIDKLMIEMDGTENKSKFGANAILGVSLAVCKAGAVEKGVPLYRHIADLAGNSEVILPVPAFNVINGGSHAGNKLAMQEFMILPVGAANFREAMRIGAEVYHNLKNVIKEKYGKDATNVGDEGGFAPNILENKEGLELLKTAIGKAGYTDKVVIGMDVAASEFFRSGKYDLDFKSPDDPSRYISPDQLADLYKSFIKDYPVVSIEDPFDQDDWGAWQKFTASAGIQVVGDDLTVTNPKRIAKAVNEKSCNCLLLKVNQIGSVTESLQACKLAQANGWGVMVSHRSGETEDTFIADLVVGLCTGQIKTGAPCRSERLAKYNQLLRIEEELGSKAKFAGRNFRNPLAK

SEQ ID NO: 4 - protein, 341 aa, Homo sapiens
  ref <308>: DATABASE ACCESSION NUMBER: Swissprot/P16220
  ref <309>: DATABASE ENTRY DATE: 2003-09-15
  ref <313>: RELEVANT RESIDUES: (1)..(341)
MTMESGAENQQSGDAAVTEAENQQMTVQAQPQIATLAQVSMPAAHATSSAPTVTLVQLPNGQTVQVHGVIQAAQPSVIQSPQVQTVQSSCKDLKRLFSGTQISTIAESEDSQESVDSVTDSQKRREILSRRPSYRKILNDLSSDAPGVPRIEEEKSEEETSAPAITTVTVPTPIYQTSSGQYIAITQGGAIQLANNGTDGVQGLQTLTMTNAAATQPGTTILQYAQTTDGQQILVPSNQVVVQAASGDVQTYQIRTAPTSTIAPGVVMASSPALPTQPAEEAARKREVRLMKNREAARECRRKKKEYVKCLENRVAVLENQNKTLIEELKALKDLYCHKSD

SEQ ID NO: 5 - protein, 254 aa, Homo sapiens
  ref <308>: DATABASE ACCESSION NUMBER: Swissprot/P18669
  ref <309>: DATABASE ENTRY DATE: 2003-09-15
  ref <313>: RELEVANT RESIDUES: (1)..(254)
MAAYKLVLIRHGESAWNLENRFSGWYDADLSPAGHEEAKRGGQALRDAGYEFDICFTSVQKRAIRTLWTVLDAIDQMWLPVVRTWRLNERHYGGLTGLNKAETAAKHGEAQVKIWRRSYDVPPPPMEPDHPFYSNISKDRRYADLTEDQLPSCESLKDTIARALPFWNEEIVPQIKEGKRVLIAAHGNSLRGIVKHLEGLSEEAIMELNLPTGIPIVYELDKNLKPIKPMQFLGDEETVRKAMEAVAAQGKAKK

SEQ ID NO: 6 - protein, 220 aa, Homo sapiens
  ref <308>: DATABASE ACCESSION NUMBER: Swissprot/P20336
  ref <309>: DATABASE ENTRY DATE: 2003-09-15
  ref <313>: RELEVANT RESIDUES: (1)..(220)
MASATDSRYGQKESSDQNFDYMFKILIIGNSSVGKTSFLFRYADDSFTPAFVSTVGIDFKVKTIYRNDKRIKLQIWDTAGQERYRTITTAYYRGAMGFILMYDITNEESFNAVQDWSTQIKTYSWDNAQVLLVGNKCDMEDERVVSSERGRQLADHLGFEFFEASAKDNINVKQTFERLVDVICEKMSESLDTADPAVTGAKQGPQLSDQQVPPHQDCAC

SEQ ID NO: 7 - protein, 206 aa, Homo sapiens
  ref <308>: DATABASE ACCESSION NUMBER: Swissprot/P11233
  ref <309>: DATABASE ENTRY DATE: 2002-06-15
  ref <313>: RELEVANT RESIDUES: (1)..(206)
MAANKPKGQNSLALHKVIMVGSGGVGKSALTLQFMYDEFVEDYEPTKADSYRKKVVLDGEEVQIDILDTAGQEDYAAIRDNYFRSGEGFLCVFSITEMESFAATADFREQILRVKEDENVPFLLVGNKSDLEDKRQVSVEEAKNRAEQWNVNYVETSAKTRANVDKVFFDLMREIRARKMEDSKEKNGKKKRKSLAKRIRERCCIL

SEQ ID NO: 8 - protein, 557 aa, Homo sapiens
  ref <308>: DATABASE ACCESSION NUMBER: Swissprot/P49023
  ref <309>: DATABASE ENTRY DATE: 2003-09-15
  ref <313>: RELEVANT RESIDUES: (1)..(557)
MDDLDALLADLESTTSHISKRPVFLSEETPYSYPTGNHTYQEIAVPPPVPPPPSSEALNGTILDPLDQWQPSGSRFIHQQPQSSSPVYGSSAKTSSVSNPQDSVGSPCSRVGEEEHVYSFPNKQKSAEPSPTVMSTSLGSNLSELDRLLLELNAVQHNPPGFPADEANSSPPLPGALSPLYGVPETNSPLGGKAGPLTKEKPKRNGGRGLEDVRPSVESLLDELESSVPSPVPAITVNQGEMSSPQRVTSTQQQTRISASSATRELDELMASLSDFKFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 9 - protein, 556 aa, Homo sapiens
  ref <308>: DATABASE ACCESSION NUMBER: Swissprot/P17987
  ref <309>: DATABASE ENTRY DATE: 2003-09-15
  ref <313>: RELEVANT RESIDUES: (1)..(556)
MEGPLSVFGDRSTGETIRSQNVMAAASIANIVKSSLGPVGLDKMLVDDIGDVTITNDGATILKLLEVEHPAAKVLCELADLQDKEVGDGTTSVVIIAAELLKNADELVKQKIHPTSVISGYRLACKEAVRYINENLIVNTDELGRDCLINAAKTSMSSKIIGINGDFFANMVVDAVLAIKYTDIRGQPRYPVNSVNILKAHGRSQMESMLISGYALNCVVGSQGMPKRIVNAKIACLDFSLQKTKMKLGVQVVITDPEKLDQIRQRESDITKERIQKILATGANVILTTGGIDDMCLKYFVEAGAMAVRRVLKRDLKRIAKASGATILSTLANLEGEETFEAAMLGQAEEVVQERICDDELILIKNTKARTSASIILRGANDFMCDEMERSLHDALCVVKRVLESKSVVPGGGAVEAALSIYLENYATSMGSREQLAIAEFARSLLVIPNTLAVNAAQDSTDLVAKLRAFHNEAQVNPERKNLKWIGLDLSNGKPRDNKQAGVFEPTIVKVKSLKFATEAAITILRIDDLIKLHPEILRIKHGSYEDAVHSGALND

SEQ ID NO: 10 - protein, 505 aa, Homo sapiens
  ref <308>: DATABASE ACCESSION NUMBER: Swissprot/P30101
  ref <309>: DATABASE ENTRY DATE: 2003-09-15
  ref <313>: RELEVANT RESIDUES: (1)..(505)
MRLRRLALFPGVALLLAAARLAAASDVLELTDDNFESRISDTGSAGLMLVEFFAPWCGHCKRLAPEYEAAATRLKGIVPLAKVDCTANTNTCNKYGVSGYPTLKIFRDGEEAGAYDGPRTADGIVSHLKKQAGPASVPLRTEEEFKKFISDKDASIVGFFDDSFSEAHSEFLKAASNLRDNYRFAHTNVESLVNEYDDNGEGIILFRPSHLTNKFEDKTVAYTEQKMTSGKIKKFIQENIFGICPHMTEDNKDLIQGKDLLIAYYDVDYEKNAKGSNYWRNRVMMVAKKFLDAGHKLNFAVASRKTFSHELSDFGLESTAGEIPVVAIRTAKGEKFVMQEEFSRDGKALERFLQDYFDGNLKRYLKSEPIPESNDGPVKVVVAENFDEIVNNENKDVLIEFYAPWCGHCKNLEPKYKELGEKLSKDPNIVIAKMDATANDVPSPYEVRGFPTIYFSPANKKLNPKKYEGGRELSDFISYLQREATNPPVIQEEKPKKKKKAQEDL

SEQ ID NO: 11 - protein, 406 aa, Homo sapiens
  ref <308>: DATABASE ACCESSION NUMBER: Swissprot/P04765
  ref <309>: DATABASE ENTRY DATE: 2003-09-15
  ref <313>: RELEVANT RESIDUES: (1)..(406)
MSASQDSRSRDNGPDGMEPEGVIESNWNEIVDSFDDMNLSESLLRGIYAYGFEKPSAIQQRAILPCIKGYDVIAQAQSGTGKTATFAISILQQIELDLKATQALVLAPTRELAQQIQKVVMALGDYMGASCHACIGGTNVRAEVQKLQMEAPHIIVGTPGRVFDMLNRRYLSPKYIKMFVLDEADEMLSRGFKDQIYDIFQKLNSNTQVVLLSATMPSDVLEVTKKFMRDPIRILVKKEELTLEGIRQFYINVEREEWKLDTLCDLYETLTITQAVIFINTRRKVDWLTEKMHARDFTVSAMHGDMDQKERDVIMREFRSGSSRVLITTDLLARGIDVQQVSLVINYDLPTNRENYIHRIGRGGRFGRKGVAINMVTEEDKRTLRDIETFYNTSIEEMPLNVADLI

SEQ ID NO: 12 - protein, 317 aa, Homo sapiens [sequence omitted]

SEQ ID NO: 13 - protein, 321 aa, Homo sapiens
  ref <308>: DATABASE ACCESSION NUMBER: Swissprot/P22087
  ref <309>: DATABASE ENTRY DATE: 2003-09-15
  ref <313>: RELEVANT RESIDUES: (1)..(321)
MKPGFSPRGGGFGGRGGFGDRGGRGGRGGFGGGRGRGGGFRGRGRGGGGGGGGGGGGGRGGGGFHSGGNRGRGRGGKRGNQSGKNVMVEPHRHEGVFICRGKEDALVTKNLVPGESVYGEKRVSISEGDDKIEYRAWNPFRSKLAAAILGGVDQIHIKPGAKVLYLGAASGTTVSHVSDIVGPDGLVYAVEFSHRSGRDLINLAKKRTNIIPVIEDARHPHKYRMLIAMVDVIFADVAQPDQTRIVALNAHTFLRNGGHFVISIKANCIDSTASAEAVFASEVKKMQQENMKPQEQLTLEPYERDHAVVVGVYRPPPKVKN

SEQ ID NO: 14 - protein, 267 aa, Homo sapiens
  ref <308>: DATABASE ACCESSION NUMBER: Swissprot/P02647
  ref <309>: DATABASE ENTRY DATE: 2003-09-15
  ref <313>: RELEVANT RESIDUES: (1)..(267)
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 15 - protein, 272 aa, Homo sapiens
  ref <308>: DATABASE ACCESSION NUMBER: Swissprot/P35232
  ref <309>: DATABASE ENTRY DATE: 2003-09-15
  ref <313>: RELEVANT RESIDUES: (1)..(272)
MAAKVFESIGKFGLALAVAGGVVNSALYNVDAGHRAVIFDRFRGVQDIVVGEGTHFLIPWVQKPIIFDCRSRPRNVPVITGSKDLQNVNITLRILFRPVASQLPRIFTSIGEDYDERVLPSITTEILKSVVARFDAGELITQRELVSRQVSDDLTERAATFGLILDDVSLTHLTFGKEFTEAVEAKQVAQQEAERARFVVEKAEQQKKAAIISAEGDSKAAELIANSLATAGDGLIELRKLEAAEDIAYQLSRSRNITYLPAGQSVLLQLPQ

SEQ ID NO: 16 - protein, 193 aa, Homo sapiens
  ref <308>: DATABASE ACCESSION NUMBER: Swissprot/P06749
  ref <309>: DATABASE ENTRY DATE: 2003-09-15
  ref <313>: RELEVANT RESIDUES: (1)..(193)
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL

SEQ ID NO: 17 - DNA, 1899 bp, Homo sapiens
  ref <308>: DATABASE ACCESSION NUMBER: GenBank/NM_012341
  ref <309>: DATABASE ENTRY DATE: 2003-04-06
  ref <313>: RELEVANT RESIDUES: (1)..(1899)
atggcacattacaacttcaagaaaattacggtggtgccgtccgccaaggacttcatagacctcacgttgtcgaagactcaacgaaagactccaaccgttattcataaacattaccaaatacatcgcattagacatttttacatgagaaaagtcaaatttactcaacagaattaccatgatagactttcacaaattctaacagatttccccaaattggatgatattcatccgttctatgctgatttgatgaatattctctacgacaaggatcattacaagttggctctggggcaaataaatattgccaaaaatttagtggacaatgttgctaaagattatgtgcgactgatgaagtatggcgactctctctaccgctgcaaacagctgaagcgtgcggccctgggacggatgtgcacagtgatcaagaggcagaagcagagtttggagtatttggagcaagtgcgtcagcatttatcccgtttgccaaccattgatccgaataccaggaccctgcttttgtgtgggtacccaaatgttgggaagtccagcttcatcaacaaggtgacgagagcagacgtggatgtccagccctatgcgttcacaaccaagtctctgtttgttgggcacatggattataagtatctacgttggcaggttgtagacactcctgggatcctggaccaccctctggaggataggaacaccatcgagatgcaggccatcactgccctggcccacctccgtgctgcggtcctgtatgtgatggatttgtctgagcagtgtgggcatgggctgagggagcagctagaactcttccagaacatcagacctctcttcatcaacaagcctctcatagttgtagccaacaaatgtgatgtgaagagaatagctgaactttctgaagatgatcagaaaatatttacagatttgcagtctgaaggattccctgtaatagagaccagcaccctgactgaggaaggtgttattaaagttaaaacagaggcttgcgataggcttttggctcatcgagtggaaaccaaaatgaagggaaataaagtgaatgaggtgctgaatagactgcacctggctatcccaaccaggagggacgataaggagaggccccctttcatccctgaaggagtggtggctcgcaggaagaggatggaaactgaggagtccaggaagaagagggaacgagatcttgagctggaaatgggagatgattatattttggatcttcagaagtactgggatttaatgaatttgtctgaaaaacatgataagataccagaaatctgggaaggccataatatagctgattatattgatccagccatcatgaagaaattggaagaattagaaaaagaagaagagctgagaacagctgctggagagtatgacagtgtatctgagagtgaagacgaagagatgctggaaatccgacagctggcaaagcaaattcgagagaaaaagaagttgaaaattctggagtccaaagaaaagaatacacagggacccaggatgccgcgaactgctaagaaggttcagaggacagttttggagaaggagatgcgtagtcttggtgttgacatggacgataaagacgatgcccattacgcagtccaggcaagaagatcccggagcatcactaggaaaagaaagcgggaagactctgctcccccgtcctctgtggcccggagtgggagttgctctcgaactccacgtgacgtttctggtcttagggatgtcaagatggtgaagaaagccaagactatgaagaatgctcagaagaagatgaatcggttggggaagaaaggggaggcggatagacacgtgtttgatatgaagcccaagcacttgctgtctgggaagaggaaagctggtaaaaaggacaggaga

SEQ ID NO: 18 - DNA, 1674 bp, Homo sapiens
  ref <308>: DATABASE ACCESSION NUMBER: GenBank/X07674
  ref <309>: DATABASE ENTRY DATE: 1994-12-16
  ref <313>: RELEVANT RESIDUES: (1)..(1674)
atgtaccgctacctgggcgaagcgctgttgctgtcccgggccgggcccgctgccctgggctcggcgtccgccgactcggccgcgttgctgggctgggcccggggacagcccgccgccgccccgcagccggggctggcattggccgcccggcgccactacagcgaggcggtggccgaccgcgaggacgaccccaacttcttcaagatggtggagggcttcttcgatcgcggcgccagcatcgtggaggacaagctggtggaggacctgaggacccgggagagcgaggagcagaagcggaaccgggtgcgcggcatcctgcggatcatcaagccctgcaaccatgtgctgagtctctccttccccatccggcgcgacgacggctcctgggaggtcatcgaaggctaccgggcccagcacagccagcaccgcacgccctgcaagggaggtatccgttacagcactgatgtgagtgtagatgaagtaaaagctttggcttctctgatgacatacaagtgtgcagtggttgatgtgccgtttgggggtgctaaagctggtgttaagatcaatcccaagaactatactgataatgaattggaaaagatcacaaggaggttcaccatggagctagcaaaaaagggctttattggtcctggcattgatgtgcctgctccagacatgagcacaggtgagcgggagatgtcctggatcgctgatacctatgccagcaccatagggcactatgatattaatgcacacgcctgtgttactggtaaacccatcagccaagggggaatccatggacgcatctctgctactggccgtggtgtcttccatgggattgaaaatttcatcaatgaagcttcttacatgagcattttaggaatgacaccagggtttggagataaaacatttgttgttcagggatttggtaatgtgggcctacactctatgagatatttacatcgttttggtgctaaatgtattgctgttggtgagtctgatgggagtatatggaatccagatggtattgacccaaaggaactggaagacttcaaattgcaacatgggtccattctgggcttccccaaggcaaagccctatgaaggaagcatcttggaggccgactgtgacatactgatcccagctgccagtgagaagcagttgaccaaatccaacgcacccagagtcaaagccaagatcattgctgaaggtgccaatgggccaacaactccagaagctgacaagatcttcctggagagaaacattatggttattccagatctctacttgaatgctggaggagtgacagtatcttactttgagtggctgaagaatctaaatcatgtcagctatggccgtttgaccttcaaatatgaaagggattctaactaccacttgctcatgtctgttcaagagagtttagaaagaaaatttggaaagcatggtggaactattcccattgtacccacggcagagttccaagacaggatatcgggtgcatctgagaaagacatcgtgcactctggcttggcatacacaatggagcgttctgccaggcaaattatgcgcacagccatgaagtataacctgggattggacctgagaacagctgcctatgttaatgccattgagaaagtcttcaaagtgtacaatgaagctggtgtgaccttcaca

SEQ ID NO: 19 - DNA, 1302 bp, Homo sapiens
  ref <308>: DATABASE ACCESSION NUMBER: GenBank/BT007163
  ref <309>: DATABASE ENTRY DATE: 2003-05-13
  ref <313>: RELEVANT RESIDUES: (1)..(1302)
atgtctattctcaagatccatgccagggagatctttgactctcgcgggaatcccactgttgaggttgatctcttcacctcaaaaggtctcttcagagctgctgtgcccagtggtgcttcaactggtatctatgaggccctagagctccgggacaatgataagactcgctatatggggaagggtgtctcaaaggctgttgagcacatcaataaaactattgcgcctgccctggttagcaagaaactgaacgtcacagaacaagagaagattgacaaactgatgatcgagatggatggaacagaaaataaatctaagtttggtgcgaacgccattctgggggtgtcccttgccgtctgcaaagctggtgccgttgagaagggggtccccctgtaccgccacatcgctgacttggctggcaactctgaagtcatcctgccagtcccggcgttcaatgtcatcaatggcggttctcatgctggcaacaagctggccatgcaggagttcatgatcctcccagtcggtgcagcaaacttcagggaagccatgcgcattggagcagaggtttaccacaacctgaagaatgtcatcaaggagaaatatgggaaagatgccaccaatgtgggggatgaaggcgggtttgctcccaacatcctggagaataaagaaggcctggagctgctgaagactgctattgggaaagctggctacactgataaggtggtcatcggcatggacgtagcggcctccgagttcttcaggtctgggaagtatgacctggacttcaagtctcccgatgaccccagcaggtacatctcgcctgaccagctggctgacctgtacaagtccttcatcaaggactacccagtggtgtctatcgaagatccctttgaccaggatgactggggagcttggcagaagttcacagccagtgcaggaatccaggtagtgggggatgatctcacagtgaccaacccaaagaggatcgccaaggccgtgaacgagaagtcctgcaactgcctcctgctcaaagtcaaccagattggctccgtgaccgagtctcttcaggcgtgcaagctggcccaggccaatggttggggcgtcatggtgtctcatcgttcgggggagactgaagataccttcatcgctgacctggttgtggggctgtgcactgggcagatcaagactggtgccccttgccgatctgagcgcttggccaagtacaaccagctcctcagaattgaagaggagctgggcagcaaggctaagtttgccggcaggaacttcagaaaccccttggccaag

SEQ ID NO: 20 - DNA, 1023 bp, Homo sapiens
  ref <308>: DATABASE ACCESSION NUMBER: GenBank/X55545
  ref <309>: DATABASE ENTRY DATE: 2000-10-04
  ref <313>: RELEVANT RESIDUES: (1)..(1023)
atgaccatggaatctggagccgagaaccagcagagtggagatgcagctgtaacagaagctgaaaaccaacaaatgacagttcaagcccagccacagattgccacattagcccaggtatctatgccagcagctcatgcaacatcatctgctcccaccgtaactctagtacagctgcccaatgggcagacagttcaagtccatggagtcattcaggcggcccagccatcagttattcagtctccacaagtccaaacagttcagtcttcctgtaaggacttaaaaagacttttctccggaacacagatttcaactattgcagaaagtgaagattcacaggagtcagtggatagtgtaactgattcccaaaagcgaagggaaattctttcaaggaggccttcctacaggaaaattttgaatgacttatcttctgatgcaccaggagtgccaaggattgaagaagagaagtctgaagaggagacttcagcacctgccatcaccactgtaacggtgccaactccaatttaccaaactagcagtggacagtatattgccattacccagggaggagcaatacagctggctaacaatggtaccgatggggtacagggcctgcaaacattaaccatgaccaatgcagcagccactcagccgggtactaccattctacagtatgcacagaccactgatggacagcagatcttagtgcccagcaaccaagttgttgttcaagctgcctctggagacgtacaaacataccagattcgcacagcacccactagcactattgcccctggagttgttatggcatcctccccagcacttcctacacagcctgctgaagaagcagcacgaaagagagaggtccgtctaatgaagaacagggaagcagctcgagagtgtcgtagaaagaagaaagaatatgtgaaatgtttagaaaacagagtggcagtgcttgaaaatcaaaacaagacattgattgaggagctaaaagcacttaaggacctttactgccacaaatcagat

SEQ ID NO: 21 - DNA, 762 bp, Homo sapiens
  ref <308>: DATABASE ACCESSION NUMBER: GenBank/J04173
  ref <309>: DATABASE ENTRY DATE: 1995-01-07
  ref <313>: RELEVANT RESIDUES: (1)..(762)
atggccgcctacaaactggtgctgatccggcacggcgagagcgcatggaacctggagaaccgcttcagcggctggtacgacgccgacctgagcccggcgggccacgaggaggcgaagcgcggcgggcaggcgctacgagatgctggctatgagtttgacatctgcttcacctcagtgcagaagagagcgatccggaccctctggacagtgctagatgccattgatcagatgtggctgccagtggtgaggacttggcgcctcaatgagcggcactatgggggtctaaccggtctcaataaagcagaaactgctgcaaagcatggtgaggcccaggtgaagatctggaggcgctcctatgatgtcccaccacctccgatggagcccgaccatcctttctacagcaacatcagtaaggatcgcaggtatgcagacctcacagaagatcagctaccctcctgtgagagtctgaaggatactattgccagagctctgcccttctggaatgaagaaatagttccccagatcaaggaggggaaacgtgtactgattgcagcccatggcaacagcctccggggcattgtcaagcatctggagggtctctctgaagaggctatcatggagctgaacctgccgactggtattcccattgtctatgaattggacaagaacttgaagcctatcaagcccatgcagtttctgggggatgaagagacggtgcgcaaagccatggaagctgtggctgcccagggcaaggccaagaag

SEQ ID NO: 22 - DNA, 660 bp, Homo sapiens
  ref <308>: DATABASE ACCESSION NUMBER: GenBank/NM_002826
  ref <309>: DATABASE ENTRY DATE: 2003-04-06
  ref <313>: RELEVANT RESIDUES: (1)..(660)
atggcatccgccacagactcgcgctatgggcagaaggagtcctcggatcagaacttcgactacatgttcaagattctcatcatcggcaacagcagcgtgggcaagacgtccttcctcttccgctatgctgacgactcgttcacgcctgccttcgtcagcaccgtgggcatcgacttcaaggtcaagaccatctatcgcaacgacaagaggatcaagctgcagatctgggacacagcagggcaagagcggtaccggaccatcaccaccgcatactaccggggcgctatgggcttcatcctcatgtatgacatcaccaacgaggaatccttcaatgcagtgcaggactggtccacccagatcaagacctactcatgggacaatgcccaggtgctgctggtaggaaacaagtgtgacatggaggatgagcgggtggtgtcatcagaacgtggccggcagctagctgaccaccttgggttcgagttctttgaggcaagcgccaaggacaacattaacgtcaagcagacctttgagcgcctggtggatgtcatctgcgagaagatgtccgagtcgttggacacggcggaccctgcggtcacaggcgccaagcagggcccacagctcagtgaccagcaggtgccaccgcaccaggactgcgcctgc

SEQ ID NO: 23 - DNA, 618 bp, Homo sapiens
  ref <308>: DATABASE ACCESSION NUMBER: GenBank/X15014
  ref <309>: DATABASE ENTRY DATE: 1993-09-12
  ref <313>: RELEVANT RESIDUES: (1)..(618)
atggctgcaaataagcccaagggtcagaattctttggctttacacaaagtcatcatggtgggcagtggtggcgtgggcaagtcagctctgactctacagttcatgtacgatgagtttgtggaggactatgagcctaccaaagcagacagctatcggaagaaggtagtgctagatggggaggaagtccagatcgatatcttagatacagctgggcaggaggactacgctgcaattagagacaactacttccgaagtggggaggggttcctctgtgttttctctattacagaaatggaatcctttgcagctacagctgacttcagggagcagattttaagagtaaaagaagatgagaatgttccatttctactggttggtaacaaatcagatttagaagataaaagacaggtttctgtagaagaggcaaaaaacagagctgagcagtggaatgttaactacgtggaaacatctgctaaaacacgagctaatgttgacaaggtattttttgatttaatgagagaaattcgagcgagaaagatggaagacagcaaagaaaagaatggaaaaaagaagaggaaaagtttagccaagagaatcagagaaagatgctgcatttta

SEQ ID NO: 24 - DNA, 1671 bp, Homo sapiens
  ref <308>: DATABASE ACCESSION NUMBER: GenBank/U14588
  ref <309>: DATABASE ENTRY DATE: 1995-03-10
  ref <313>: RELEVANT RESIDUES: (1)..(1671)
atggacgacctcgacgccctgctggcggacttggagtctaccacctcccacatctccaaacggcctgtgttcttgtcggaggagaccccctactcatacccaactggaaaccacacataccaggagattgccgtgccaccccccgtccccccacccccgtccagcgaggccctcaatggcacaatccttgaccccttagaccagtggcagcccagcggctcccgattcatccaccagcagcctcagtcctcatcacctgtgtacggctccagtgccaaaacttccagtgtctccaaccctcaggacagtgttggctctccgtgctcccgagtgggtgaggaggagcacgtctacagcttccccaacaagcagaaatcagctgagccttcacccaccgtaatgagcacgtccctgggcagcaacctttctgaactcgaccgcctgctgctggaactgaacgctgtacagcataacccgccaggcttccctgcagatgaggccaactcaagccccccgcttcctggggccctgagccccctctatggtgtcccagagactaacagccccttgggaggcaaagctgggcccctgacgaaagagaagcctaagcggaatgggggccggggcctggaggacgtgcggcccagtgtggagagtctcttggatgaactggagagctccgtgcccagccccgtccctgccatcactgtgaaccagggcgagatgagcagcccgcagcgcgtcacctccacccaacagcagacacgcatctcggcctcctctgccaccagggagctggacgagctgatggcttcgctgtcggatttcaagttcatggcccaggggaagacagggagcagctcaccccctggggggcccccgaagcccgggagccagctggacagcatgctggggagcctgcagtctgacctgaacaagctgggggtcgccacagtcgccaaaggagtctgcggggcctgcaagaagcccatcgccgggcaggttgtgaccgccatggggaagacgtggcaccccgagcacttcgtctgcacccactgccaggaggagatcggatcccggaacttcttcgagcgggatggacagccctactgtgaaaaggactaccacaacctcttctccccgcgctgctactactgcaacggccccatcctggataaagtggtgacagcccttgaccggacgtggcaccctgaacacttcttctgtgcacagtgtggagccttctttggtcccgaagggttccacgagaaggacggcaaggcctactgtcgcaaggactacttcgacatgttcgcacccaagtgtggcggctgcgcccgggccatcctggagaactatatctcagccctcaacacgctgtggcatcctgagtgctttgtgtgccgggaatgcttcacgccattcgtgaacggcagcttcttcgagcacgacgggcagccctactgtgaggtgcactaccacgagcggcgcggctcgctgtgttctggctgccagaagcccatcaccggccgctgcatcaccgccatggccaagaagttccaccccgagcacttcgtctgtgccttctgcctcaagcagctcaacaagggcaccttcaaggagcagaacgacaagccttactgtcagaactgcttcctcaagctcttctgc

SEQ ID NO: 25 - DNA, 1668 bp, Homo sapiens
  ref <308>: DATABASE ACCESSION NUMBER: GenBAnk/NM_030752
  ref <309>: DATABASE ENTRY DATE: 2003-04-06
  ref <313>: RELEVANT RESIDUES: (1)..(1668)
atggaggggcctttgtccgtgttcggtgaccgcagcactggggaaacgatccgctcccaaaacgttatggctgcagcttcgattgccaatattgtaaaaagttctcttggtccagttggcttggataaaatgttggtggatgatattggtgatgtaaccattactaacgatggtgcaaccatcctgaagttactggaggtagaacatcctgcagctaaagttctttgtgagctggctgatctgcaagacaaagaagttggagatggaactacttcagtggttattattgcagcagaactcctaaaaaatgcagatgaattagtcaaacagaaaattcatcccacatcagttattagtggctatcgacttgcttgcaaggaagcagtgcgttatatcaatgaaaacctaattgttaacacagatgaactgggaagagattgcctgattaatgctgctaagacatccatgtcttccaaaatcattgggataaatggtgatttctttgctaacatggtagtagatgctgtacttgctattaaatacacagacataagaggccagccacgctatccagtcaactctgttaatattttgaaagcccatgggagaagtcaaatggagagtatgctcatcagtggctatgcactcaactgtgtggtgggatcccagggcatgcccaagagaatcgtaaatgcaaaaattgcttgccttgacttcagcctgcaaaaaacaaaaatgaagcttggtgtacaggtggtcattacagaccctgaaaaactggaccaaattagacagagagaatcagatatcaccaaggagagaattcagaagatcctggcaactggtgccaatgttattctaaccactggtggaattgatgatatgtgtctgaagtattttgtggaggctggtgctatggcagttagaagagttttaaaaagggaccttaaacgcattgccaaagcttctggagcaactattctgtcaaccctggccaatttggaaggtgaagaaacttttgaagctgcaatgttgggacaggcagaagaagtggtacaggagagaatttgtgatgatgagctgatcttaatcaaaaatactaaggctcgtacgtctgcatcgattatcttacgtggggcaaatgatttcatgtgtgatgagatggagcgctctttacatgatgcactttgtgtagtgaagagagttttggagtcaaaatctgtggttcccggtgggggtgctgtagaagcagccctttccatataccttgaaaactatgcaaccagcatggggtctcgggaacagcttgcgattgcagagtttgcaagatcacttcttgttattcccaatacactagcagttaatgctgcccaggactccacagatctggttgcaaaattaagagcttttcataatgaggcccaggttaacccagaacgtaaaaatctaaaatggattggtcttgatttgagcaatggtaaacctcgagacaacaaacaagcaggggtgtttgaaccaaccatagttaaagttaagagtttgaaatttgcaacagaagctgcaatcaccattcttcgaattgatgatcttattaaattacatccagaaatccttcggattaaacatggaagttatgaagatgctgttcactctggagcccttaatgat

SEQ ID NO: 26 - DNA, 1515 bp, Homo sapiens
  ref <308>: DATABASE ACCESSION NUMBER: GenBank/NM_005313
  ref <309>: DATABASE ENTRY DATE: 2003-04-06
  ref <313>: RELEVANT RESIDUES: (1)..(1515)
atgcgcctccgccgcctagcgctgttcccgggtgtggcgctgcttcttgccgcggcccgcctcgccgctgcctccgacgtgctagaactcacggacgacaacttcgagagtcgcatctccgacacgggctctgcgggcctcatgctcgtcgagttcttcgccccctggtgtggacactgcaagagacttgcacctgagtatgaagctgcagctaccagattaaaaggaatagtcccattagcaaaggttgattgcactgccaacactaacacctgtaataaatatggagtcagtggatatccaaccctgaagatatttagagatggtgaagaagcaggtgcttatgatggacctaggactgctgatggaattgtcagccacttgaagaagcaggcaggaccagcttcagtgcctctcaggactgaggaagaatttaagaaattcattagtgataaagatgcctctatagtaggttttttcgatgattcattcagtgaggctcactccgagttcctaaaagcagccagcaacttgagggataactaccgatttgcacatacgaatgttgagtctctggtgaacgagtatgatgataatggagagggtatcatcttatttcgtccttcacatctcactaacaagtttgaggacaagactgtggcatatacagagcaaaaaatgaccagtggcaaaattaaaaagtttatccaggaaaacatttttggtatctgccctcacatgacagaagacaataaagatttgatacagggcaaggacttacttattgcttactatgatgtggactatgaaaagaacgctaaaggttccaactactggagaaacagggtaatgatggtggcaaagaaattcctggatgctgggcacaaactcaactttgctgtagctagccgcaaaacctttagccatgaactttctgattttggcttggagagcactgctggagagattcctgttgttgctatcagaactgctaaaggagagaagtttgtcatgcaggaggagttctcgcgtgatgggaaggctctggagaggttcctgcaggattactttgatggcaatctgaagagatacctgaagtctgaacctatcccagagagcaatgatgggcctgtgaaggtagtggtagcagagaattttgatgaaatagtgaataatgaaaataaagatgtgctgattgaattttatgccccttggtgtggtcactgtaagaacctggagcccaagtataaagaacttggcgagaagctcagcaaagacccaaatatcgtcatagccaagatggatgccacagccaatgatgtgccttctccatatgaagtcagaggttttcctaccatatacttctctccagccaacaagaagctaaatccaaagaaatatgaaggtggccgtgaattaagtgattttattagctatctacaaagagaagctacaaacccccctgtaattcaagaagaaaaacccaagaagaagaagaaggcacaggaggatctc

SEQ ID NO: 27 - DNA, 1218 bp, Homo sapiens
  ref <308>: DATABASE ACCESSION NUMBER: GenBank/NM_001416
  ref <309>: DATABASE ENTRY DATE: 2003-04-07
  ref <313>: RELEVANT RESIDUES: (1)..(1218)
atgtctgcgagccaggattcccgatccagagacaatggccccgatgggatggagcccgaaggcgtcatcgagagtaactggaatgagattgttgacagctttgatgacatgaacctctcggagtcccttctccgtggcatctacgcctatggttttgagaagccctctgccatccagcagcgagccattctaccttgtatcaagggttatgatgtgattgctcaagcccaatctgggactgggaaaacggccacatttgccatatcgattctgcagcagattgaattagatctaaaagccacccaggccttggtcctagcacccactcgagaattggctcagcagatacagaaggtggtcatggcactaggagactacatgggcgcctcctgtcacgcctgtatcgggggcaccaacgtgcgtgctgaggtgcagaaactgcagatggaagctccccacatcatcgtgggtacccctggccgtgtgtttgatatgcttaaccggagatacctgtcccccaaatacatcaagatgtttgtactggatgaagctgacgaaatgttaagccgtggattcaaggaccagatctatgacatattccaaaagctcaacagcaacacccaggtagttttgctgtcagccacaatgccttctgatgtgcttgaggtgaccaagaagttcatgagggaccccattcggattcttgtcaagaaggaagagttgaccctggagggtatccgccagttctacatcaacgtggaacgagaggagtggaagctggacacactatgtgacttgtatgaaaccctgaccatcacccaggcagtcatcttcatcaacacccggaggaaggtggactggctcaccgagaagatgcatgctcgagatttcactgtatccgccatgcatggagatatggaccaaaaggaacgagacgtgattatgagggagtttcgttctggctctagcagagttttgattaccactgacctgctggccagaggcattgatgtgcagcaggtttctttagtcatcaactatgaccttcccaccaacagggaaaactatatccacagaatcggtcgaggtggacggtttggccgtaaaggtgtggctattaacatggtgacagaagaagacaagaggactcttcgagacattgagaccttctacaacacctccattgaggaaatgcccctcaatgttgctgacctcatc

SEQ ID NO: 28 - DNA, 951 bp, Homo sapiens
  ref <308>: DATABASE ACCESSION NUMBER: GenBank/NM_006098
  ref <309>: DATABASE ENTRY DATE: 2003-04-07
  ref <313>: RELEVANT RESIDUES: (1)..(951)
atgactgagcagatgacccttcgtggcaccctcaagggccacaacggctgggtaacccagatcgctactaccccgcagttcccggacatgatcctctccgcctctcgagataagaccatcatcatgtggaaactgaccagggatgagaccaactatggaattccacagcgtgctctgcggggtcactcccactttgttagtgatgtggttatctcctcagatggccagtttgccctctcaggctcctgggatggaaccctgcgcctctgggatctcacaacgggcaccaccacgaggcgatttgtgggccataccaaggatgtgctgagtgtggccttctcctctgacaaccggcagattgtctctggatctcgagataaaaccatcaagctatggaataccctgggtgtgtgcaaatacactgtccaggatgagagccactcagagtgggtgtcttgtgtccgcttctcgcccaacagcagcaaccctatcatcgtctcctgtggctgggacaagctggtcaaggtatggaacctggctaactgcaagctgaagaccaaccacattggccacacaggctatctgaacacggtgactgtctctccagatggatccctctgtgcttctggaggcaaggatggccaggccatgttatgggatctcaacgaaggcaaacacctttacacgctagatggtggggacatcatcaacgccctgtgcttcagccctaaccgctactggctgtgtgctgccacaggccccagcatcaagatctgggatttagagggaaagatcattgtagatgaactgaagcaagaagttatcagtaccagcagcaaggcagaaccaccccagtgcacttccctggcctggtctgctgatggccagactctgtttgctggctacacggacaacctggtgcgagtgtggcaggtgaccattggcacacgc

SEQ ID NO: 29 - DNA, 963 bp, Homo sapiens
  ref <308>: DATABASE ACCESSION NUMBER: genBank/NM_001436
  ref <309>: DATABASE ENTRY DATE: 2003-04-03
  ref <313>: RELEVANT RESIDUES: (1)..(963)
atgaagccaggattcagtccccgtgggggtggctttggcggccgagggggctttggtgaccgtggtggtcgtggaggccgagggggctttggcgggggccgaggtcgaggcggaggctttagaggtcgtggacgaggaggaggtggaggcggcggcggcggtggaggaggaggaagaggtggtggaggcttccattctggtggcaaccggggtcgtggtcggggaggaaaaagaggaaaccagtcggggaagaatgtgatggtggagccgcatcggcatgagggtgtcttcatttgtcgaggaaaggaagatgcactggtcaccaagaacctggtccctggggaatcagtttatggagagaagagagtctcgatttcggaaggagatgacaaaattgagtaccgagcctggaaccccttccgctccaagctagcagcagcaatcctgggtggtgtggaccagatccacatcaaaccgggggctaaggttctctacctcggggctgcctcgggcaccacggtctcccatgtctctgacatcgttggtccggatggtctagtctatgcagtcgagttctcccaccgctctggccgtgacctcattaacttggccaagaagaggaccaacatcattcctgtgatcgaggatgctcgacacccacacaaataccgcatgctcatcgcaatggtggatgtgatctttgctgatgtggcccagccagaccagacccggattgtggccctgaatgcccacaccttcctgcgtaatggaggacactttgtgatttccattaaggccaactgcattgactccacagcctcagccgaggccgtgtttgcctccgaagtgaaaaagatgcaacaggagaacatgaagccgcaggagcagttgacccttgagccatatgaaagagaccatgccgtggtcgtgggagtgtacaggccaccccccaaggtgaagaac

SEQ ID NO: 30 - DNA, 801 bp, Homo sapiens
  ref <308>: DATABASE ACCESSION NUMBER: GenBank/NM_000039
  ref <309>: DATABASE ENTRY DATE: 2003-04-03
  ref <313>: RELEVANT RESIDUES: (1)..(801)
atgaaagctgcggtgctgaccttggccgtgctcttcctgacggggagccaggctcggcatttctggcagcaagatgaacccccccagagcccctgggatcgagtgaaggacctggccactgtgtacgtggatgtgctcaaagacagcggcagagactatgtgtcccagtttgaaggctccgccttgggaaaacagctaaacctaaagctccttgacaactgggacagcgtgacctccaccttcagcaagctgcgcgaacagctcggccctgtgacccaggagttctgggataacctggaaaaggagacagagggcctgaggcaggagatgagcaaggatctggaggaggtgaaggccaaggtgcagccctacctggacgacttccagaagaagtggcaggaggagatggagctctaccgccagaaggtggagccgctgcgcgcagagctccaagagggcgcgcgccagaagctgcacgagctgcaagagaagctgagcccactgggcgaggagatgcgcgaccgcgcgcgcgcccatgtggacgcgctgcgcacgcatctggccccctacagcgacgagctgcgccagcgcttggccgcgcgccttgaggctctcaaggagaacggcggcgccagactggccgagtaccacgccaaggccaccgagcatctgagcacgctcagcgagaaggccaagcccgcgctcgaggacctccgccaaggcctgctgcccgtgctggagagcttcaaggtcagcttcctgagcgctctcgaggagtacactaagaagctcaacacccag

SEQ ID NO: 31 - DNA, 816 bp, Homo sapiens
  ref <308>: DATABASE ACCESSION NUMBER: GenBank/NM_000039
  ref <309>: DATABASE ENTRY DATE: 2003-04-03
  ref <313>: RELEVANT RESIDUES: (1)..(816)
  ref <308>: DATABASE ACCESSION NUMBER: GenBank/BT007411
  ref <309>: DATABASE ENTRY DATE: 2003-03-13
  ref <313>: RELEVANT RESIDUES: (1)..(816)
atggctgccaaagtgtttgagtccattggcaagtttggcctggccttagctgttgcaggaggcgtggtgaactctgccttatataatgtggatgctgggcacagagctgtcatctttgaccgattccgtggagtgcaggacattgtggtaggggaagggactcattttctcatcccgtgggtacagaaaccaattatctttgactgccgttctcgaccacgtaatgtgccagtcatcactggtagcaaagatttacagaatgtcaacatcacactgcgcatcctcttccggcctgtcgccagccagcttcctcgcatcttcaccagcatcggagaggactatgatgagcgtgtgctgccgtccatcacaactgagatcctcaagtcagtggtggctcgctttgatgctggagaactaatcacccagagagagctggtctccaggcaggtgagcgacgaccttacagagcgagccgccacctttgggctcatcctggatgacgtgtccttgacacatctgaccttcgggaaggagttcacagaagcggtggaagccaaacaggtggctcagcaggaagcagagagggccagatttgtggtggaaaaggctgagcaacagaaaaaggcggccatcatctctgctgagggcgactccaaggcagctgagctgattgccaactcactggccactgcaggggatggcctgatcgagctgcgcaagctggaagctgcagaggacatcgcgtaccagctctcacgctctcggaacatcacctacctgccagcggggcagtccgtgctcctccagctgccccag

SEQ ID NO: 32 - DNA, 579 bp, Homo sapiens
  ref <308>: DATABASE ACCESSION NUMBER: GenBank/AF498970
  ref <309>: DATABASE ENTRY DATE: 2002-05-01
  ref <313>: RELEVANT RESIDUES: (1)..(579)
atggctgccatccggaagaaactggtgattgttggtgatggagcctgtggaaagacatgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtgggaagaaaaaatctggttgccttgtcttg

SEQ ID NO: 33 - protein, 175 aa, Homo sapiens
MDIAIHHPWIRRPFFPFHSPSRLFDQFFGEHLLESDLFPTSTSLSPFYLRPPSFLRAPSWFDTGLSEMRLEKDRFSVNLDVKHFSPEELKVKVLGDVIEVHGKHEERQDEHGFISREFHRKYRIPADVDPLTITSSLSSDGVLTVNGPRKQVSGPERTIPITREEKPAVTAAPKK

(19) United States
(12) Patent Application Publication
(10) Pub. No.: US 2007/0122810 A1
(43) Pub. Date: May 31, 2007

(54) Brain tumor marker and method of diagnosing brain tumor

(75) Inventors: Yasuo Iwadate, Sakura-shi Chiba (JP); Takaki Hiwasa, Chiba (JP); Masaki Takiguchi, Chiba (JP); Akira Yamaura, Chiba (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Japan Science And Technology Agency, Kawaguchi-shi, Saitama (JP)

(21) Appl. No.: 10/569,732

(22) PCT Filed: Apr. 30, 2004
(86) PCT No.: PCT/JP04/06345

§ 371 Date: Feb. 28, 2006

(30) Foreign Application Priority Data
Sep. 1, 2003 (JP) 2003-309356

(51) Int. Cl. C12Q 1/68; G01N 33/574; C07H 21/04; C07K 14/82

(52) U.S. Cl. 435/6; 435/7.23; 435/69.1; 435/320.1; 435/325; 530/350; 536/23.5; 536/24.3

(57) ABSTRACT

The present invention provides a brain tumor marker, characterized in that the brain tumor marker is at least one protein expressed in a brain tumor tissue and selected from the group of proteins having the amino acid sequences represented by SEQ ID NOS: 1 to 16, respectively, wherein the protein is expressed in at least one tissue selected from the group of the tissues of a highly malignant brain tumor, a low malignant brain tumor, a drug-sensitive brain tumor, and a drug-resistant brain tumor; a polynucleotide encoding the brain tumor marker; and a method of diagnosing brain tumors by using the brain tumor marker or the polynucleotide as an indicator.

### BACKGROUND OF THE INVENTION

1. Technical Field

The invention of the present application relates to a marker protein useful for diagnosis of human brain tumors with respect to grade of malignancy and drug sensitivity or resistance, a polynucleotide encoding the marker protein, and a method of cancer diagnosis for brain tumors by using the marker protein or the polynucleotide.

2. Background Art

Astrocytoma is diversified so widely as to make it very difficult how it should be classified pathologically. Even if astrocytomas are of a similar low grade, some of them may progress into brain tumor (glioblastoma), while others may remain in a silent state over years. Some genetic abnormalities and variation in gene expression levels have been found to be associated with tumor formation of astrocytoma and its aggravation of malignancy (Cavenee, W. K. et al., “Diffuse infiltrating astrocytomas”, In: W. K. Cavenee and P. Kleihues (eds.), pp. 9-15, Lyon: IARC Press, 2000), but these events have had a limited utility in molecular biological diagnosis of glioma. Establishment of a diagnostic method based on molecular biology to predict the prognosis and therapeutic response of patients requires a more comprehensive characterization of such gene expressions as would be correlated to clinical behaviors of glioma. Molecular biological diagnosis of human glioma as described above will finally lead to a target-specific therapeutic approach to develop a therapeutic strategy for individual patients and thus enhance clinical therapeutic effects on the whole patients.

In several types of human cancer, molecular expressions have been profiled using microarrays based on oligonucleotides or cDNAs to obtain molecular markers useful for making a choice among the therapies (Alizadeh, A. A. et al., Nature 403: 503-11, 2000; and Bittner, M. et al., Nature 406: 536-540, 2000). The profiles of gene expression created using the microarray technique have been examined to reveal that many genes are expressed differentially among various histologic types or grades of glioma in the brain (Pomeroy, S. L. et al., Nature 415: 436-42, 2002; Rickman, D. S. et al., Cancer Res. 61: 6885-6891, 2001; and Sallinen, S. L. et al., Cancer Res. 60: 6617-22, 2000). However, since biological systems involve transcriptional and posttranscriptional control, posttranslational modification, and protein migration between different cell compartments, such characteristics cannot be identified by microarray systems on the level of RNA. Consequently, some molecular biological diagnoses have also been proposed, wherein their targets are proteins expressed specifically in tumor tissues including brain tumors (Japanese Patent Publication (Kokai) No. 2002-223765; and Japanese Patent Publication (Kohyo) Nos. 2003-506100, 2002-519702 and 2002-509734).

The molecular biological method of diagnosis using protein markers expressed specifically in brain tumor tissues, as described above, has been indicated to be effective to diagnose brain tumors in their early stage, and a number of novel protein markers have been proposed for the method. In order to improve the method in diagnostic accuracy, however, it is critical to provide many protein markers which reflect the biological characteristics of brain tumors or the like, and use the protein markers in combination.

It is an object of the invention of the present application to provide a novel protein marker useful for a more accurate diagnosis of brain tumors, in view of the circumstances as described above.

It is another object of the invention of the present application to provide a genetic material encoding the protein marker, an antibody against the protein marker, and a method of diagnosing brain tumors using the protein marker or antibody.

### SUMMARY OF THE INVENTION

The present application provides the following inventions (1) to (19) in order to solve the above problems:
(1) a brain tumor marker, characterized in that the brain tumor marker is at least one protein expressed in a brain tumor tissue and selected from the group of proteins having the amino acid sequences represented by SEQ ID NOS: 1 to 16, respectively, wherein the protein is expressed in at least one tissue selected from the group of the tissues of a highly malignant brain tumor, a low malignant brain tumor, a drug-sensitive brain tumor, and a drug-resistant brain tumor;
(2) a highly malignant brain tumor marker, wherein the brain tumor marker is at least one protein selected from the group of proteins expressed in a highly malignant brain tumor tissue, wherein the protein has the amino acid sequence represented by SEQ ID NO: 1, 4, 5, 6 or 7;
(3) a low malignant brain tumor marker, wherein the brain tumor marker is at least one protein selected from the group of proteins expressed in a low malignant brain tumor tissue, wherein the protein has the amino acid sequence represented by SEQ ID NO: 2 or 3;
(4) a drug-sensitive brain tumor marker, wherein the brain tumor marker is at least one protein selected from the group of proteins expressed in a drug-sensitive brain tumor tissue, wherein the protein has the amino acid sequence represented by SEQ ID NO: 5, 8, 11, 13, 14, 15 or 16;
(5) a drug-resistant brain tumor marker, wherein the brain tumor marker is at least one protein selected from the group of proteins expressed in a drug-resistant brain tumor tissue, wherein the protein has the amino acid sequence represented by SEQ ID NO: 1, 2, 9, 10, 12 or 16;
(6) an antibody which binds to a protein selected from the group of proteins having the amino acid sequences represented by SEQ ID NOS: 1 to 16, respectively;
(7) a diagnostic kit, characterized in that the diagnostic kit comprises at least one of the antibody according to the invention (6) and/or a labeled antibody derived therefrom;
(8) a polynucleotide encoding the highly malignant brain tumor marker according to the invention (2);
(9) a polynucleotide encoding the low malignant brain tumor marker according to the invention (3);
(10) a polynucleotide encoding the drug-sensitive brain tumor marker according to the invention (4);
(11) a polynucleotide encoding the drug-resistant brain tumor marker according to the invention (5);
(12) a DNA fragment consisting of a partial contiguous sequence of any one of SEQ ID NOS: 17 to 32;
(13) a DNA microarray comprising the polynucleotide according to any one of the inventions (8) to (11) and/or the DNA fragment according to the invention (12);
(14) a set of primers for amplifying by PCR any one of the nucleotide sequences of SEQ ID NOS: 17 to 32;
(15) a method of diagnosing a highly malignant brain tumor, characterized in that a biological sample from a subject is assayed for the expression level of the polynucleotide according to the invention (8), wherein the subject is judged to be a patient with a highly malignant brain tumor if the subject has a higher expression level of at least one said polynucleotide than a healthy person;
(16) a method of diagnosing a low malignant brain tumor, characterized in that a biological sample from a subject is assayed for the expression level of the polynucleotide according to the invention (9), wherein the subject is judged to be a patient with a low malignant brain tumor if the subject has a higher expression level of at least one said polynucleotide than a healthy person;
(17) a method of diagnosing a drug-sensitive brain tumor, characterized in that a biological sample from a subject is assayed for the expression level of the polynucleotide according to the invention (10), wherein the subject is judged to be a patient with a drug-sensitive brain tumor if the subject has a higher expression level of at least one said polynucleotide than a healthy person;
(18) a method of diagnosing a drug-resistant brain tumor, characterized in that a biological sample from a subject is assayed for the expression level of the polynucleotide according to the invention (11), wherein the subject is judged to be a patient with a drug-resistant brain tumor if the subject has a higher existence level of at least one said polynucleotide than a healthy person; and
(19) a method of diagnosing a brain tumor, characterized in that said method combines the respective methods according to the inventions (15) to (18), wherein a brain tumor which a subject suffers from is judged to be one selected from the group of:
(a) highly malignant and drug-sensitive;
(b) highly malignant and drug-resistant;
(c) low malignant and drug-sensitive; and
(d) low malignant and drug-resistant.

### BRIEF DESCRIPTION OF THE DRAWINGS

FIG. 1 shows representative two-dimensional electrophoresis images of normal brain tissue (A), astrocytoma (B), undifferentiated astrocytoma (C) and multiform glioblastoma (D);

FIG. 2 shows 4 different protein spots on two-dimensional electrophoresis gel, wherein the spots were of proteins expressed differently between low grade astrocytoma and high grade astrocytoma (multiform glioblastoma or undifferentiated astrocytoma);

FIG. 3 shows a diagram of hierarchical clustering applied on each of the cases based on proteome profiling data determined for 75 glioblastoma samples and 10 normal brain samples. The hierarchical grade was classified into 4 groups consisting of normal brain tissue (Normal), astrocytoma (A), undifferentiated astrocytoma (AA) and multiform glioblastoma (GM). Survival time of each patient is shown in the right column with a limit of 5 years at most;

FIG. 4 shows the spots of proteins differently expressed between low grade astrocytoma and high grade astrocytoma (FIG. 4A), the results of analyses by matrix-associated laser desorption/ionization time-of-flight mass spectrometry (MALDI-TOF MS) for the expressed proteins (FIG. 4B, (I) and (II)), and the determined amino acid sequence (FIG. 4C), respectively. The determined amino acid sequence is shown in FIG. 4C by underlining on the full length sequence of protein (α-crystallin);

FIG. 5 shows specific examples of drug sensitivity test for representative anti-cancer agents. CPM and ACNU are effective in A; CPM, ACNU and TAX are effective in B; VCR is effective in C; and D is an example in which no drug is effective. In the phylogenetic tree in sample axis, A and B are located at sites approximate to normal brain tissue, C is located at a site slightly away from normal brain tissue, and D is located at a site more far-off from normal brain tissue; and

FIG. 6 shows a diagram of hierarchical clustering applied on each of the cases based on the proteome profiling data determined for 75 glioblastoma samples and 10 normal brain samples as well as 8 samples of oligodendroglioma which is known to be comparatively high in drug sensitivity. The hierarchical grade was classified into 5 groups consisting of normal brain tissue (Normal), astrocytoma (Astro), undifferentiated astrocytoma (AA), multiform glioblastoma (GM) and oligodendroglioma (Oligo). Sensitivity to 10 representative drugs (CPM, ACNU, CDDP, PEP, ADM, VP-16, CA, TAX and VCR) for which dose response relationship was evaluated is shown in the right column.

### DETAILED DESCRIPTION OF THE INVENTION

The inventors of this application have investigated proteome profiling patterns among plural samples of human brain tumor using two-dimensional electrophoresis and matrix-associated laser desorption/ionization time-of-flight (MALDI-TOF) mass spectrometry and identified 16 kinds of proteins (SEQ ID NOS: 1-16) as shown in Table 1.

TABLE 1

| | | Amino acid | | | |
|---|---|---|---|---|---|
| | | sequence | | cDNA sequence | |
| | | | Sequence | | Sequence |
| No. | Name of Protein | Swissprot | Listing | GenBank | Listing |
| 1 | Nucleolar GTP-binding protein | Q9BZE4 | 1 | NM_012341 | 17 |
| 2 | Glutamate dehydrogenase 1 | O00367 | 2 | X07674 | 18 |
| 3 | Phosphopyruvate hydratase | P06733 | 3 | BT007163 | 19 |
| 4 | cAMP response element-binding protein | P16220 | 4 | X55545 | 20 |
| 5 | Phosphoglycerate mutase 1 | P18669 | 5 | J04173 | 21 |
| 6 | Rab3A, member Ras oncogene family | P20336 | 6 | NM_002826 | 22 |
| 7 | Ras-related protein Ral-A | P11233 | 7 | X15014 | 23 |
| 8 | Paxillin | P49023 | 8 | U14588 | 24 |
| 9 | T complex protein 1, alpha subunit | P17987 | 9 | NM_030752 | 25 |
| 10 | Protein disulphide isomerase A3 | P30101 | 10 | NM_005313 | 26 |
| | Eucaryotic translation initiation factor | P04765 | 11 | NM_001416 | 27 |
| | 4A(p37) | | | | |
| 12 | Activated C kinase receptor | P25388 | 12 | NM_006098 | 28 |
| 13 | Fibrillarin | P22087 | 13 | NM_001436 | 29 |
| 14 | Apolipoprotein A-1 | P02647 | 14 | NM_000039 | 30 |
| 15 | Prohibitin | P35232 | 15 | NM_000039 | 31 |
| 16 | Transforming protein RhoA | P06749 | 16 | AF498970 | 32 |

And the inventors have further confirmed that these 16 kinds of proteins express in either one or more tissues of highly malignant brain tumor, low malignant brain tumor, drug sensitive brain tumor and drug resistant brain tumor. That is, the inventors have found that the proteins Nos. 1, 4, 5, 6 and 7 in Table 1 express in highly malignant brain tumor tissue and can be markers of highly malignant brain tumor; the proteins Nos. 2 and 3 express in low malignant brain tumor tissue and can be markers of low malignant brain tumor; the proteins Nos. 5, 8, 11, 13, 14, 15 and 16 express in drug sensitive brain tumor tissue and can be markers of drug sensitive brain tumor; and the proteins Nos. 1, 2, 9, 10, 12 and 16 express in drug resistant brain tumor tissue and can be markers of drug resistant brain tumor.

As for the proteins No. 1-16 in Table 1, the following functions are known.

No. 1: This is present in the nucleolus and has an amino acid sequence homologous to the other G protein groups and participates in intracellular signal transduction through G protein-coupled receptors present in the cell membrane.

No. 2: It is known that various kinds of metabolic enzyme activities are generally enhanced in some cancer cells.

No. 3: Isoenzyme (NSE) specifically present in nerve cells is known, and it is considered to be reduced with decrease of normal nerve cells.

No. 4: This is a transcription factor, reportedly has important roles in the fields such as inflammatory reaction and neuranagenesis and participates in transcription of target genes at the final step of signal transduction system from tyrosine kinase type receptors, which play important roles in canceration of various kinds of cells.

No. 5: This is an enzyme participating in the metabolism of phosphoglycerate generated in glycolysis, and availability as a disease severity marker in cerebral apoplexy and myocardial infarction and increase in activity in some cancers have been reported.

No. 6: This belongs to Rab family among G proteins, and is observed to be expressed in brain and endocrine tissues. It is considered that this is important for the formation of secretory granules in synapses and the like. There are few reports that pointed out association with tumorigenesis.

No. 7: This belongs to Ras family among G proteins, and it is considered to participate in various kinds of diseases including cancer.

No. 8: This participates in cell adhesion and also participates in signal transduction through the action on cell surface receptors and cytoskeleton.

No. 9: This is a molecular chaperon important for the structure formation of cytoplasm proteins such as actin and tubulin, and association with large bowel cancer and a hepatocellular carcinoma has been pointed out.

No. 10: This is located in endoplasmic reticula and functions as a molecular chaperon for the protein synthesized there.

No. 11: This binds to RNA, and is a protein which controls the initiation of translation.

No. 12: This interacts with protein kinase C and participates in cell adhesion and movement through integrin. This also has cell proliferation inducing action in certain cancer cells.

No. 13: This is a protein which is present in endoplasmic reticula and interacts with RNA and has chaperon function.

No. 14: This participates in excretion of lipid from cells, and the reduction thereof can be a risk factor of ischemic heart disease along with HDL. In the meantime, lipid component is essential to cell membrane synthesis, and it is known that the level thereof decreases in fast proliferating cells.

No. 15: This is a molecular chaperon localized in the membrane of mitochondria. Suppressive effect on cell proliferation is also known, and this is a protein having cancer suppressive effect.

No. 16: This belongs to Rho family among G proteins, and it is known to participate in regulating cytoskeleton as well as in malignant transformation of cells. In late years, it is reported that the expression thereof decreases as malignancy grade of brain tumor elevates.

However, as for these 16 kinds of proteins, correlation with high/low malignancy and/or drug sensitivity/resistance of brain tumor has not been known at all.

The invention of this application is based on new marker proteins and polynucleotides encoding the same as described above.

Here, in the present invention, “protein” and “peptide” mean a molecule consisting of plural amino acid residues linked by an amide bond (peptide bond) with each other. “Polynucleotide” refers to a molecule having 100 or more linked phosphate esters of nucleosides (ATP, GTP, CTP, UTP; or dATP, dGTP, dCTP, dTTP) in which purine or pyrimidine binds to sugar through β-N-glycoside bond, and “oligonucleotide” refers to a molecule in which 2-99 such phosphate esters of nucleosides are linked.

In addition, as for the nucleotide and amino acid sequences shown in Table 1, addition, deletion, or substitution for other bases of one or more bases (in other words, one or more mutations), or addition, deletion, or substitution for other amino acids of one or more amino acids based on such mutations of bases (in other words, one or more mutations) are also included.

Furthermore, “highly malignant tumor” means a tumor which has high proliferation capability and invasive ability and becomes fatal at early stage. Therefore, “highly malignant tumor” and “the low malignant tumor” in the present invention can be specifically distinguished by the criteria that the case where the survival time is within one and half years due to high proliferation capability and invasive ability is judged as “high malignancy” while the case where the survival time is two or three or more years is judged as “low malignancy”.

In addition, “drug sensitivity” means that there is an anti-cancer agent which can induce cell death in such a blood concentration as obtained by usual clinical dosage and “drug resistance” means that there is no such anti-cancer agents at the time point when the the present invention is completed. The drug sensitive brain tumor means brain tumor cells which will be annihilated by or the proliferation capability of which will be eliminated or reduced by, or the invasive ability of which will be eliminated or reduced by anti-cancer agents, for example, alkylating agents (4-hydroperoxy-cyclophosphamide (CPM) (1 μg/ml), 4-hydroperoxy-ifosfamide (IFOS) (1 μg/ml), melphalan (MPL) (0.5 μg/ml), carboquone (CQ) (0.1 μg/ml), nimustine (ACNU) (2 μg/ml), ranimustine (MCNU) (2 μg/ml), cisplatinum (CDDP) (0.5 μg/ml) and carboplatin (JM-8) (4 μg/ml)); antimetabolites (methotrexate (MTX) (3 μg/ml), 5-fluorouracil (5-FU) (10 μg/ml), thioinosine (6-MRP) (3 μg/ml) and cytosine arabinoside (CA) (4 μg/ml)); antibiotics (mitomycin C (MMC) (0.2 μg/ml), bleomycin (BLM) (1 μg/ml), peplomycin (PEP) (0.5 μg/ml), chromomycin A3 (TM) (0.01 μg/ml) and neocarsinostatin (NCS) (0.15 μg/ml)); topoisomerase inhibitor (actinomycin D(ACD) (0.01 μg/ml), aclarubicin (ACR) (0.6 μg/ml), adriamycin (ADM) (0.3 μg/ml), daunomycin (DM) (0.6 μg/ml), pirarubicin (THP) (0.3 μg/ml),epirubicin (4′-EPI) (0.4 μg/ml), mitoxantrone (MIT) (0.06 μg/ml), etoposide (VP-16) (3 μg/ml) and camptothecin (CPT-11) (3 μg/ml)); microtubule inhibitor (vincristine (VCR) (0.1 μg/ml), vinblastin (VLB) (0.1 μg/ml), vindesin (VDS) (0.1 μg/ml) and paclitaxel (TAX) (0.6 μg/ml)).

In addition, “diagnosis” in the present invention means a judgment whether a subject suffers from brain tumor, a judgment whether there is a risk that a subject will suffer from brain tumor in the future, a judgment on the effect of treatment and a judgment whether there is a risk that brain tumor will relapse after treatment. The diagnosis also includes determination of degree and risk of brain tumor.

Other terms and concept in the present invention will be prescribed in detail in the description on the embodiments of the invention and Examples. In addition, various techniques to be used to carry out the present invention can be conducted readily and with certainty by those skilled in the art based on known documents, etc. except for such techniques as specifically shown with references. For example, techniques of gene engineering and molecular biology can be performed by a method of described in or a method described in a reference cited in or a method substantially the same as or modified from the followng: J. Sambrook, E. F. Fritsch & T. Maniatis, “Molecular Cloning: A Laboratory Manual (2nd edition)”, Cold Spring Harbor Laboratory Press, Cold Spring Harbor, N.Y. (1989); D. M. Glover et al. ed., “DNA Cloning”, 2nd ed., Vol. 1 to 4, (The Practical Approach Series), IRL Press, Oxford University Press (1995); Ausubel, F. M. et al., Current Protocols in Molecular Biology, John Wiley & Sons, New York, N.Y, 1,995; Japanese Biochemical Society ed., “Zoku Seikagaku Jikken Kouza 1, Idenshi Kenkyu-hou II”(“Sequel Biochemical Experiment Series 1, Methods for Gene Study II”), Tokyo Kagaku Dojin (1986); Japanese Biochemical Society ed., “Shin Seikagaku Jikken Kouza 2, Kakusan III (Kumikae DNA Gijutsu)” (“New Biochemical Experiment Series 2, Nucleic Acid III” (Recombinant DNA Technique)”), Tokyo Kagaku Dojin (1986); R. Wu ed., “Methods in Enzymology”, Vol. 68 (Recombinant DNA), Academic Press, New York (1980); R. Wu et al. ed., “Methods in Enzymology”, Vol. 100 (Recombinant DNA, Part B) & 101 (Recombinant DNA, Part C), Academic Press, New York (1983); R. Wu et al. ed., “Methods in Enzymology”, Vol. 153 (Recombinant DNA, Part D), 154(Recombinant DNA, Part E) & 155 (Recombinant DNA, Part F), Academic Press, New York (1987); J. H. Miller ed., “Methods in Enzymology”, Vol. 204, Academic Press, New York (1991); R. Wu et al. ed., “Methods in Enzymology”, Vol. 218, Academic Press, New York (1993); S. Weissman (ed.), “Methods in Enzymology”, Vol. 303, Academic Press, New York (1999); J. C. Glorioso et al. (ed.) “Methods in Enzymology”, Vol. 306, Academic Press, New York (1999), etc.

According to the invention of this application, tumor marker proteins to be used for simply and accurately judging high/low grade of malignancy of brain tumor and/or presence/absence of drug sensitivity are provided. Various molecular biological materials relating to these marker proteins are also provided and the grade of malignancy and/or drug sensitivity of brain tumor can be diagnosed precisely by using these, and thereby selection of an effective therapeutic method is enabled.

### BEST MODE FOR CARRYING OUT THE INVENTION

The brain tumor marker according to the invention (1) is each of proteins of Nos. 1-16 shown in Table 1. These proteins are characterized in that they have amino acid sequences represented by SEQ ID NOS: 1-16 respectively, each protein being expressed in at least one tissue of a highly malignant brain tumor, a low malignant brain tumor, a drug sensitive brain tumor and a drug resistant brain tumor.

These brain tumor markers are classified into groups of the following inventions (2) to (5).

The brain tumor markers according to the invention (2) are protein Nos. 1, 4, 5, 6 and 7 shown in Table 1 (SEQ ID NOS: 1,4, 5, 6 and 7, respectively), which are highly malignant tumor marker proteins expressed in a highly malignant brain tumor.

The brain tumor markers according to the invention (3) are protein Nos. 2 and 3 shown in Table 1 (SEQ ID NOS: 2 and 3, respectively), and they are low malignant brain tumor markers expressed in low malignant brain tumor tissues.

Furthermore, the brain tumor markers according to the invention (4) are protein Nos. 5, 8, 11, 13, 14, 15 and 16 shown in Table 1 (SEQ ID NOS: 5, 8, 11, 13, 14, 15 and 16, respectively), and they are drug sensitive brain tumor markers expressed in drug sensitive brain tumor tissues.

Furthermore, the brain tumor markers according to the invention (5) are protein Nos. 1, 2, 9, 10, 12 and 16 shown in Table 1 (SEQ ID NOS: 1, 2, 9, 10, 12 and 16, respectively), and they are drug resistant brain tumor markers expressed in drug resistant brain tumor tissues.

The marker proteins according to these inventions (1) to (5) can be used, for example, as antigens for producing antibodies according to the invention (6) using purified products thereof, respectively.

The purified product of each marker protein can be obtained by a method comprising isolating and purifying them from a human cell (for example, cell from brain tumor tissue), or can be obtained by a genetic engineering method using a polynucleotide (cDNA) consisting of a nucleotide sequence shown in each of SEQ ID NOS: 17-32. For example, RNA may be prepared by in vitro transcription from a recombination expression vector carrying each cDNA and may be expressed in vitro by performing in vitro translation using the cDNA as a template. They can be expressed in a transformed cell prepared by introducing a recombinant expression vector into a procaryotic cell such as E. coli or Bacillus subtilis or a eucaryotic cell such as yeast, insect cell or mammalian cell.

When the marker protein is expressed by in vitro translation, a recombination expression vector may be prepared by inserting a polynucleotide into a vector having an RNA polymerase promoter and adding this vector to an in vitro translation system such as rabbit reticular cell lysate or wheat germ extract including RNA polymerase corresponding to the promoter, and the protein of interest can be produced in vitro. Examples of RNA polymerase promoters include T7, T3 and SP6. Examples of vectors including such RNA polymerase promoter include pKA 1, pCDM8, pT3/T7 18, pT7/3 19 and pBluescript II.

When the marker protein is expressed in microorganism such as E. coli, an expression vector, in which a vector having origin, promoter, ribosome-binding site, DNA cloning site, terminator, etc. and replicable in the microorganism is recombined with a polynucleotide, may be prepared, and the protein which the polynucleotide encodes can be expressed in the microorganism by transforming a host cell with this expression vector and culturing the obtained transformant, wherein it can be expressed as a protein fused with another protein. Examples of expression vectors for E. coli include pUC system, pBluescript II, pET expression system and pGEX expression system.

When the marker protein is expressed in an eucaryotic cell, a recombinant vector may be prepared by inserting a polynucleotide into an expression vector for eucaryotic cell having promoter, splicing region, poly (A) additional site and so on, and the protein of interest can be expressed in a transformed eucaryotic cell by introducing the recombinant vector into an eucaryotic cell. Examples of expression vectors include pKA1, pCDM8, pSVK3, pMSG, pSVL, pBK-CMV, pBK-RSV, EBV vector, pRS, pcDNA3, pMSG and pYES2. The protein can be also expressed as a fusion protein to which any of various tags such as His tag, FLAG tag, myc tag, HA tag, and GFP is added when pIND/V5-His, pFLAG-CMV-2, pEGFP-N1, pEGFP-C1 or the like is used as an expression vector. Generally, for eucaryotic cells, cultured mammalian cells such as monkey kidney cell COS7, Chinese hamster ovary cell CHO, budding yeast, fission yeast, silkworm cell, and Xenopus oocyte can be used, but any kind of eucaryotic cell is usable as long as it can express the marker proteins of the present invention. In order to introduce the expression vector into the eucaryotic cell, well-known methods such as electroporation, calcium phosphate method, liposome method, and DEAE dextran method can be used.

Conventional separation procedures may be used in combination for isolating and purifying the peptide of interest from the culture after expressing the marker protein in a procaryotic or eucaryotic cell. Examples of such methods include treatment with a denaturant or surfactant such as urea, ultrasonication, enzymatic digestion, salting out and solvent sedimentation method, dialysis, centrifugation, ultrafiltration, gel filtration, SDS-PAGE, isoelectric focusing, ion exchange chromatography, hydrophobic chromatography, affinity chromatography, and reverse phase chromatography.

The recombinant protein obtained by the above-mentioned method can be fused with any other protein in order to obtain a fusion protein. Examples thereof include fusion proteins with glutathione-S-transferase (GST) or green fluorescence protein (GFP). Furthermore, the protein expressed in the transformed cell may be subjected to various modifications in the cell after it was translated. Therefore, modified proteins are included in the scope of the protein of the present invention. Examples of such a post-translational modification include elimination of N-terminal methionine, N-terminal acetylation, sugar chain addition, limited proteolysis by intracellular protease, myristoylation, isoprenylation, and phosphorylation.

The antibody of invention (6) is a polyclonal antibody or a monoclonal antibody, and includes any of the whole antibody molecule and Fab, F(ab′)₂ and Fv fragments thereof, which can bind to an epitope of each of the proteins having amino acid sequences of SEQ ID NOS: 1-16. Such an antibody can be obtained, for example, from the blood serum after immunizing an animal with the protein or a fragment thereof as an immunogen in case of polyclonal antibody. Alternatively, the antibody can be prepared by introducing an expression vector for eucaryotic cell as mentioned above into the muscle or skin of an animal by injection or by a gene gun and then collecting the blood serum. As the animal, mouse, rat, rabbit, goat, or chicken can be used.

The monoclonal antibody can be prepared following known methods for preparing a monoclonal antibody (“Monoclonal Antibody”, Komei Hasemune and Hiroshi Terada, Hirokawa Shoten, 1990; “Monoclonal Antibody” James W. Goding, third edition, Academic Press, 1996).

The antibody of the present invention (6) includes an antibody labeled with a labeling substance. Enzyme, radioisotope or fluorochrome can be used as a labeling substance. There is not particular limitation on the enzyme as long as it satisfies conditions such as a large turnover number, stability in binding to the antibody, and specific staining of the substrate, and enzymes used for normal EIA include, for example, peroxidase, β-galactosidase, alkaline phosphatase, glucose oxidase, acetylcholine esterase, glucose-6-phosphate dehydrogenase, malate dehydrogenase, etc. Enzyme inhibitor, coenzyme or the like can be also used. Binding of such an enzyme to an antibody can be performed by well-known methods using a cross-linking agent such as maleimide compound. Known substances can be used depending on the kind of enzyme to be used. For example, when peroxidase is used as an enzyme, 3,3′, 5,5′- tetramethylbenzene can be used and when alkaline phosphatase is used as an enzyme, p-nitrophenol, etc. can be used. Examples of the radioisotope, include those conventionally used in RIA such as ¹²⁵I and ³H. For fluorochrome, those used in normal fluorescent antibody techniques such as fluorescein isothiocyanate (FITC) and tetramethylrhodamine isothiocyanate (TRITC) can be used.

The invention (7) is a brain tumor diagnostic kit including one or more kind of the above antibody and/or labeled antibody thereof Such a diagnostic kit may include an antibody or a labeled antibody in the liquid phase or may have an antibody or a labeled antibody bound to the solid phase support. Furthermore, when the label of labeled antibody is an enzyme, this diagnostic kit may include the substrate therefor, for example, and when a solid phase support is included, a solution for washing to remove molecules not bound to the solid phase may be included. Various types of such kits are commercially available depending on the type of the analyte, and the diagnostic kit of the present invention can be composed of each element used for a conventionally known or used kit except that an antibody and/or a labeled antibody provided by the present invention is used.

Inventions (8) to (11) are polynucleotides (i.e., DNA fragments, or RNA fragments) encoding the marker proteins of the inventions (2) to (5), respectively. Specifically, they are genomic DNAs encoding each protein, mRNA transcripted from the genomic DNA, and cDNA synthesized from the MRNA. The cDNAs have nucleotide sequences of SEQ ID NOS: 17-32, respectively. They may be single-stranded or double-stranded. Furthermore, the sense and antisense strands of the genomic DNA, mRNA, or cDNA are also included. Still further, in the case of genomic DNA, expression controlling region (e.g., promoter, enhancer, or suppressor region) are included.

These polynucleotides can be readily obtained by a well-known method, respectively. For example, the cDNAs can be synthesized by using a well-known method (Mol. Cell Biol.2, 161-170, 1982; J. Gene 25, 263-269, 1983; Gene, 150, 243-250, 1994) and can be obtained by isolating each cDNA using probe DNAs prepared based on the respective nucleotide sequences from SEQ ID NOS: 1-16. The obtained cDNA can be amplified by, for example, usually performed gene amplification methods such as PCR (Polymerase Chain Reaction) method, NASBN (Nucleic acid sequence based amplification) method, TMA (Transcription-mediated amplification) method and SDA (Strand Displacement Amplification) method. Each cDNA can also be obtained in a required amount by RT-PCR method using primer sets as provided by the present invention and isolated mRNA from a human cell as a template.

The invention (12) is directed to DNA fragments consisting of each of partial contiguous sequences of SEQ ID NOS: 17-32. In addition, DNA fragments consisting of partial contiguous sequences of the sequences complementary to SEQ ID NOS 17-32 are also included in the DNA fragment. Preferably it is a DNA fragment of 6 to 75 nucleotides, more preferably 10 to 40 nucleotides, and most preferably 15 to 30 nucleotides.

This DNA fragment can be obtained, for example, by cleaving the above-mentioned polynucleotide (cDNA) with a suitable restriction enzyme. Alternatively, it can be synthesized in vitro by well-known chemosynthesis techniques described in Carruthers (1982) Cold Spring Harbor Symp. Quant. Biol.47:411-418; Adams (1983) J. Am. Chem. Soc. 105:661; Belousov (1997) Nucleic Acid Res. 25:3440-3444, Frenkel (1995) Free Radic. Biol. Med. 19:373-380; Blommers (1994) Biochemistry 33:7886-7896; Narang (1979) Meth.Enzymol. 68:90; Brown (1979) Meth. Enzymol. 68:109; Beaucage (1981) Tetra.Lett.22:1,859 and U.S. Pat. No. 4,458,066. This DNA fragment can be used as a probe by labeling with a labeling substance as in the above-mentioned polynucleotide. Labeling can be performed by radioisotope (RI) method or non-RI method, but it is preferable to use non-RI method. The non-RI method includes fluorescent labeling methods, biotin labeling methods, chemiluminescence methods and the like, but it is preferable to use fluorescent labeling methods. As for the fluorescent substances, those which can be bound to the basic part of the oligonucleotide can be suitably selected and used but cyanine coloring agent (for example, Cy3 or Cy5 of Cy Dye™ series), rhodamine 6G reagent, N-acetoxy-N²-acetylaminofluorene (AAF), AAIF (iodine derivative of AAF) and the like can be used.

Furthermore, DNA fragment of the invention (12) is characterized in that the fragment hybridizes under stringent condition with a polynucleotide of the above-described inventions (8) to (11). The stringent condition as used herein is a condition which enables the selective and detectable specific binding between the above-mentioned polynucleotide and DNA fragment probe. The stringent condition is defined with salt concentration, organic solvent (for example, formamide), temperature and other well-known conditions. In other words, the stringency increases as the salt concentration is reduced, the organic solvent concentration is increased or the hybridization temperature is elevated. For example, stringent salt concentration is usually about 750 mM or less NaCl and about 75 mM or less trisodium citrate, more preferably about 500 mM or less NaCl and about 50 mM or less trisodium citrate, most preferably about 250 mM or less NaCI and about 25 mM or less trisodium citrate. Stringent organic solvent concentration is about 35% or more and most preferably about 50% or more of formamide. The stringent temperature condition is about 30° C. or more, more preferably about 37° C. or more and most preferably about 42° C. or more. The other conditions include hybridization time, concentration of detergent (for example, SDS), and presence/absence of carrier DNA, etc., and various stringencies can be set by combining these conditions.

According to one preferable embodiment, hybridization is performed under the condition of 750 mM NaCl, 75 mM trisodium citrate and 1% SDS at a temperature of 30° C. In a more preferable embodiment, hybridization is performed under the condition of 500 mM NaCl, 50 mM trisodium citrate, 1% SDS, 35% formamide and 100 μg/ml of denatured salmon sperm DNA at a temperature of 37° C. In the most preferable embodiment, hybridization is performed under the condition of 250 mM NaCl, 25 mM trisodium citrate, 1% SDS, 50% formamide and 200 μg/ml of denatured salmon sperm DNA at a temperature of 42° C. Washing condition after hybridization also influences the stringency. This washing condition is also defined with salt concentration and temperature, and the stringency of washing is increased by decreasing the salt concentration and elevating the temperature. For example, the salt condition which is stringent for washing is preferably about 30 mM or less NaCl and about 3 mM or less trisodium citrate, most preferably about 15 mM or less NaCl and about 1.5 mM or less trisodium citrate. The temperature condition which is stringent for washing is about 25° C. or more, more preferably about 42° C. or more and most preferably about 68° C. or more. According to one preferable embodiment, washing is performed under the condition of 30 mM NaCl, 3 mM trisodium citrate and 0.1% SDS at a temperature of 25° C. In more preferable embodiment, washing is performed under the condition of 15 mM NaCl, 1.5 mM trisodium citrate, 0.1% SDS at a temperature of 42° C. In the most preferable embodiment, washing is performed under the condition of 15 mM NaCI, 1.5 mM trisodium citrate, 0.1% SDS at a temperature of 68° C.

The invention (13) is a DNA microarray comprising a polynucleotide(s) of the above-described inventions (8) to (11) and/or a DNA fragment(s) of the above-described invention (14).

Methods used for the preparation of microarray are methods of directly synthesizing an oligonucleotide on solid-phase carrier surface (on-chip synthesis), and methods of fixing an oligonucleotide or polynucleotide prepared beforehand on a solid phase carrier surface. The microarray to be used in the present invention can be prepared by either of these methods. The on-chip synthesis can be performed, for example, by a method of selectively synthesizing at a predetermined minute area on the matrix by combination of the use of a protection group which can be selectively removed by radiation of light, with the photolithography technology used in the production of semiconductor and the solid phase synthesis technology (masking technology: for example, Fodor, S.P.A. Science 251:767, 1991). On the other hand, when an oligonucleotide or polynucleotide prepared beforehand is fixed on solid-phase carrier surface, the oligonucleotide or a polynucleotide to which a functional group has been bonded is synthesized and spotted to covalently bond to the solid phase carrier surface which has been subjected to surface treatment (for example, Lamture, J.B. et al. Nucl.Acids Res. 22:2121-2125, 1994; Guo, Z. et al. Nucl.Acids Res. 22:5456-5465, 1994).

The oligonucleotide or polynucleotide is generally covalently bonded to the surface-treated solid phase carrier through a spacer or a cross linker. There is also a known method in which minute pieces of polyacrylamide gel are aligned on the glass surface to which synthetic oligonucleotides are covalently bonded (Yershov, G. et al. Proc. Natl. Acad. Sci. USA 94:4913, 1996). There is also another known method in which an array of microelectrodes is formed on a silica microarray and a permeable agarose layer containing streptavidin is set on the electrodes as a reactive site, biotinylated oligonucleotides are fixed on the site by positively charging this site, and the charge is controlled on the site, thereby enabling high-speed and precise hybridization (Sosnowski, R.G. et al. Proc. Natl. Acad. Sci. USA 94:1,119-1123, 1997).

The Invention (14) is a primer set for amplifying the nucleotide sequences of SEQ ID NOS: 17 to 32 by PCR.

These primer sets can be designed based on each of he nucleotide sequences of SEQ ID NOS: 17-32 and can be prepared through the synthesis and purification steps. As kept in mind upon the primer design, the following can be pointed out for example. The size (the number of bases) of a primer is 15-40 bases, desirably 15-30 bases in consideration of satisfying specific annealing between the primer and the template DNA. However, at least 30 bases are effective when LA (long accurate) PCR is performed. Complementary sequences between the both primers should be avoided so that a set of or a pair (2 strands) of primers consisting of sense (at 5′-end side) and antisense (at 3′-end side) strands may not anneal with each other, and a self-complementary sequence should be also avoided to prevent the formation of hairpin structure in the primers. Furthermore, GC content should be about 50% to secure stable binding to the template DNA, and GC-rich or AT-rich regions should not be unevenly distributed within the primers. Because the annealing temperature depends on Tm (melting temperature), primers having Tm values in the range of 55 to 65° C. and similar to each other are to be selected in order to obtain highly specific PCR products. In addition, it is necessary to note that the final concentration of the primers used in PCR should be adjusted to about 0.1 to about 1 μM. Commercially available softwares for primer designing, for example, Oligo™ [product of National Bioscience Inc. (U.S.A.)], GENETYX [product of Software Development Co., Ltd. (Japan)] can be also used.

With materials provided by each of the above-described inventions, diagnosis of brain tumor, particularly judgment and diagnosis of the characteristics of the brain tumor (high/low grade of malignancy, presence/absence of drug sensitivity) are enabled.

The method of the inventions (15) to (18) is a method for diagnosing brain tumor using as an indicator the expression levels of the polynucleotides of the above-described inventions (8) to (11), respectively. Specifically, the invention (15) is a method for diagnosing highly malignant brain tumor using as an indicator the expression level of the polynucleotide of the invention (8); the invention (16) is a method for diagnosing low malignant brain tumor using as an indicator the expression level of the polynucleotide of the invention (9); the invention (17) is a method for diagnosing drug sensitive brain tumor using as an indicator the expression level of the polynucleotide of the invention (10); and the invention (18) is a method for diagnosing drug resistant brain tumor using as an indicator the expression level of the polynucleotide of the invention (11). The “polynucleotide” in this method is a genomic gene DNA encoding each of the above-mentioned tumor markers and means the one which is present in a tissue cell isolated from a subject. A “subject” is, for example, a patient who has received a medical examination and suspected to be with brain tumor by MRI inspection or the like, and the “biological sample” is an excised tissue from the brain, blood or the like of the patient. As a specific criteria, the level of polynucleotide expression of a subject relative to that of a healthy person is higher by 10% or more, preferably 30% or more, more preferably 70% or more, and most preferably 100% or more.

The expression level of the polynucleotide can be known by measuring transcription product (mRNA, protein) of the polynucleotide. For example, assay of transcription product is possible by in situ hybridization, Northern blotting, dot blotting, RNase protection assay, RT-PCR, Real-Time PCR (for example, Journal of Molecular Endocrinology, 25, 169-193, 2000 and references cited there), DNA microarray analysis method (for example, Mark Shena, ed., “Microarray Biochip Technology”, Eaton Publishing, 2000), etc.

For example, the level of mRNA transcripted from a polynucleotide can be measured by northern blotting assay using a probe obtained by labeling the DNA fragment of the above-described invention (12), as an intensity of the label signal.

The level of the polynucleotide expression can be measured simply and extensively by using DNA microarray of the above-described invention (13). Specifically, cDNA may be synthesized, for example, using mRNA isolated from a cell of the subject as a template, and may be amplified by PCR. Labeled dNTP may be incorporated to form a labeled cDNA. The labeled cDNA is contacted with the macroarray, and the label signal is detected as an indicator of cDNA hybridized with a capture probe (oligonucleotide or polynucleotide) on the microarray. Hybridization can be carried out by dispensing an aqueous liquid of labeled cDNA to a 96-well or 384-well plastic plate for spotting onto the microarray. The spotted amount is around 1 to 100 nl. The hybridization is preferably performd in the range of room temperature to 70° C. for 6 to 20 hours. It is preferable to wash with a mixture of detergent and buffer after the hybridization is completed. Example of the buffer used are citrate buffer, phosphate buffer, borate buffer, Tris buffer, Good's buffer, etc., preferably citrate buffer.

Alternatively, the transcription product mRNA of the polynucleotide can be also measured by the RT-PCR method using a primer set of the invention (15). Further, the level of the polynucleotide mRNA can be quantified by using the quantitative RT-PCR method.

In addition, when the protein (or tumor marker) is measured as a transcription product of the polynucleotide, the measurement can be performed using the antibody of the above-described invention (6) or the diagnostic kit of the above-described invention (7). Such methods using an antibody include, for example, immunostaining, such as tissue staining or cell staining, immunoelectron microscopy, and immunoassay. such as competitive immunoassay or non-competitive immunoassay. Alternatively, radioimmunoassay (RIA), fluoroimmunoassay (FIA), luminescent immunoassay (LIA), enzyme immunoassay (EIA), ELISA and the like can also be used. B-F separation may or may not be performed before measurement. Preferable methods are RIA, EIA, FIA, and LIA, or sandwich assay. The sandwich assay may be simultaneous sandwich type assay, forward sandwich type assay, reverse sandwich type assay, or the like.

One aspect of such a diagnosis method using an antibody is a method of detecting the binding between the antibody and the brain tumor marker protein in a liquid phase system. For example, the labeled antibody of the invention (6) are contacted with a biological sample to bind the labeled antibody to the marker protein, and their conjugate is separated. Separation can be performed by methods of separating the conjugate of marker protein and labeled antibody, for example conventional separation means such as chromatography method, solid phase method, etc. Known conventional Western blotting method can also be used. When enzyme is used as a label, the label signal is determined by adding a substrate which is decomposed by action of the enzyme to develop a color, determining the enzymatic activity by optically measuring a decomposed amount of the substrate and converting it into the amount of bound antibody, and calculating an amount of the antibody through comparison with standard values. When radioisotope is used, the amount of the radiation emitted by the radioisotope is measured with a scintillation counter or the like. In addition, when fluorochrome is used, the amount of fluorescence can be measured with a measuring apparatus combined with a fluorescence microscope.

Another diagnostic method in a liquid phase system comprises contacting the antibody (as a primary antibody) of the invention (6) with a biological sample to bind the primary antibody to HRF protein, thereby forming a conjugate; binding a labeled antibody (as a secondary antibody) to the conjugate; and detecting the label signal from the conjugate of these three entities. Alternatively, non-labeled secondary antibody may be bound to the antibody and antigen peptide conjugate at first and subsequently a labeling substance may be bound to the secondary antibody in order to further enhance the signal. The binding of the labeling substance to the secondary antibody can be performed, for example, by biotinylating a secondary antibody while avidinating the labeling substance. Alternatively, third antibody recognizing a part of the region of the secondary antibody (for example, Fc region) may be labeled in order to bind it to the secondary antibody. Monoclonal antibodies can be used for both of the primary antibody and secondary antibody, or either one of the primary antibody and secondary antibody may be a polyclonal antibody. The separation of the conjugate from the liquid phase and the detection of the signal can be performed as described above.

Another aspect using an antibody is a method of testing binding of the antibody to the marker protein in a solid-phase system. Any method using solid-phase system is preferable in view of detection of an extremely small amount of the marker protein or in view of simplicity and easiness of the operation. In other words, in the solid-phase system, the antibody of the invention (6) is immobilized on a resin plate, membrane or the like, a marker protein is bound to this immobilized antibody, non-bound protein is washed out, after that, a labeled antibody is bound to the antibody and marker protein conjugate left on the plate, thereby detecting a signal from this labeled antibody. This method is a so-called “sandwich method,” and when enzyme is used as a marker, this method is widely used as “ELISA” (enzyme linked immunosorbent assay). As for the two kinds of antibodies, monoclonal antibodies may be used for the both of them, or either one of them may be a polyclonal antibody.

The method of the invention (19) is a diagnosis method to judge which of the following brain tumors:
(a) highly malignant and drug sensitive;
(b) highly malignant and drug resistant;
(c) low malignant and drug sensitive; and
(d) low malignant and drug resistant
is the brain tumor of a subject by combining the diagnosis methods of the above-described inventions (15) to (18).

This method is extremely useful in the selection of therapeutic methods and so on because the grade of malignancy and the presence/absence of the drug sensitivity can be diagnosed at the same time. For example, when the tumor is judged as a highly malignant but drug sensitive, anti-cancer agent therapy having sensitivity can be selected. When the brain tumor is judged as a highly malignant and drug resistant, appropriate therapy such as radiotherapy or removal of a cancerous part can be selected.

The invention of this application will be described in more detail and more specifically by Examples but the invention is not limited by the following Examples.

### EXAMPLES

### Example 1

Protein Markers Associated with the Malignancy of Brain Tumor were Identified.

(1)Materials and Methods

(1-1) Tissue Sample

75 human glioma tissues and noncancerous brain tissues surgically removed were used. Upon use of the tissues, an informed consent document was obtained from a patient or a guardian beforehand. 52 samples were diagnosed as multiform glioblastoma (GM, grade IV), 13 samples as undifferentiated astrocytoma (AA, grade III) and 10 samples as astrocytoma (grade II) according to the WHO standard. 10 normal brain samples were obtained from patients who received excision of off-axis brain tumor or an epilepsy operation. The excised tumors or the normal brain samples were divided into two parts immediately after they were surgically excised. One part of them was fixed in 10% formaldehyde and embedded in paraffin for histopathological diagnosis. The other part was snap-frozen in liquid nitrogen and stored at −80° C. for extracting protein. The protocol of this experimental research was approved by Institutional Review Board aimed at analyzing a proteome profiling pattern in glioma.

(1-2) Preparation of Sample for Two-dimensional Gel

The frozen samples were mechanically homogenized in a lysis buffer containing 30 mM Tris-HCl (pH 7.5), 150 mM NaCl, 1% Triton X-100, 10% glycerol and a protease inhibitor cocktail to obtain a protein lysis liquid. Bradford assay using BioRad Protein Assay (Japan BioRad Company, Tokyo) was carried and the concentration of the total proteins were determined and standardized to 1 μg/μl for all samples. Each protein sample (100 μg) was freeze-dried and a sample buffer containing 7M urea, 2M thiourea, 4%(w/v) CHAPS and 1%(w/v) DTT was added thereto.

(1-3) Two-dimensional Gel Electrophoresis

Isoelectric focusing (IEF) for the first dimension separation of proteins was carried out using Multiphore II electrophoresis system (Amersham Biosciences, N.J., U.S.A.) and Investigator 5,000V two-dimension power supply (Genomic Solutions, Inc., Ann Arbor, Mich., U.S.A.) following the instructions of the manufacturers. An Immobiline Dry IPG strip (7cm, pH 3 to 10 non-linear IPG strip) produced by Amersham Biosciences company was swollen in with a mixed solution of sample/swelling buffer overnight. The strip was subjected to electrophoresis using a ramping IPG strip (200 to 5000V) electrophoresis algorithm attaining the final electrophoresis voltage of 3,500V and 3700Vh. Following IEF, the gel strip was equilibrated in an SDS equilibration buffer and subjected to electrophoresis in a vertical SDS-PAGE Slab gel containing 12.5% acrylamide. Some sample were subjected to electrophoresis with molecular weight (Mr) and isoelectric point (pI) calibration markers for calculating the molecular weight and isoelectric point of protein spots at image analysis. Immeadiately after the two-dimensional SDS-PAGE, the gel was stained with Silver Quest Ag Staining Kit (Invitrogen Co., Ltd., Tokyo) compatible with mass spectrometry. (1-4) Image analysis of two-dimensional gel The Ag stains on the gel were digitized using a flatbed image scanner with a resolution of 300 dpi. Location and determination of the intensity of protein spots and matching of the spot between the paired gels were performed using Phoretix 2D Advanced software (Ver 5.01, Nonlinear Dynamic, Ltd., New Castle, U.K.). Spot matching was automatically carried out after manually identifying the common anchor spots. Matching was performed for gel images obtained from 10 normal brain tissue samples and a composite reference image including most of the possible spots was prepared. The gel test in duplicate for 10 normal brain samples was performed and reproducibility of the experiment was confirmed. Only the spots which were present/completely absent between the tumor and control gels were assumed to be altered spots. This strict standard was used in this research so that possibility of the later validation would be the highest throughout the de-regulated proteins. (1-5) MALDI-TOF mass spectrometry

Protein spots were excised from the gel and treated with ZipTip C18 (Millipore Co., Ltd., USA). The digestion in the gel was carried out in an enzyme solution containing 50 mM NH₄HCO₃, 5mM CaCl₂ and 12.5 ng/l trypsin. An aliquot of the purified sample was spotted on the matrix crystal of α-cyano-4-hydroxylcinnamic acid on the stainless steel target and air-dried. The determination of mass was carried out by AXIMA-CFR mass spectrometer (Shimadzu Corporation, Kyoto, Japan) equipped with a nitrogen laser (λ=337 mm) and delayed extraction. The laser desorbed cations were analyzed after having accelerated at 20 kV in linear mode. The outer calibration was carried out with a normal oligonucleotide mixture. The proteins were identified by PMF method using Mascot Search (Matrix Science, Ltd., London, U.K.) on the Web.

(1-6) Statistic Analysis

Hierarchical cluster analysis was performed on the tumor and normal tissue samples. Identification analysis was carried out to find out proteins which had an influence on the histologic grade significantly. All the statistical analysis was carried out with SPSS for Windows (registered trademark) software (SPSS, Inc., Chicago, Ill., U.S.A.).

(2) Results

(2-1) Proteome Profiling Pattern in Normal Brain Tissue and Glioma

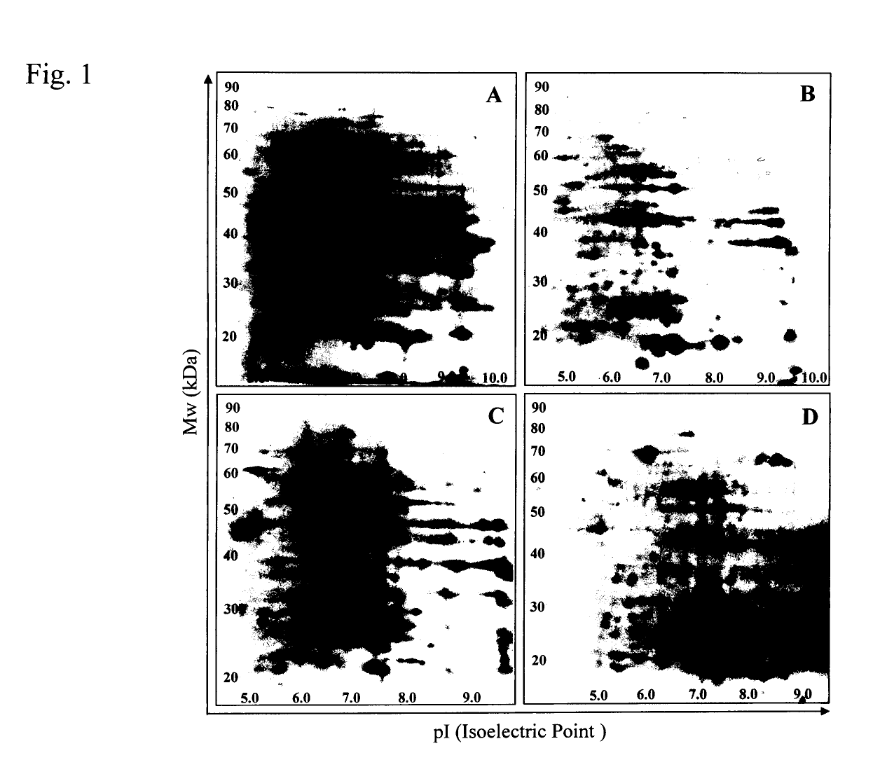

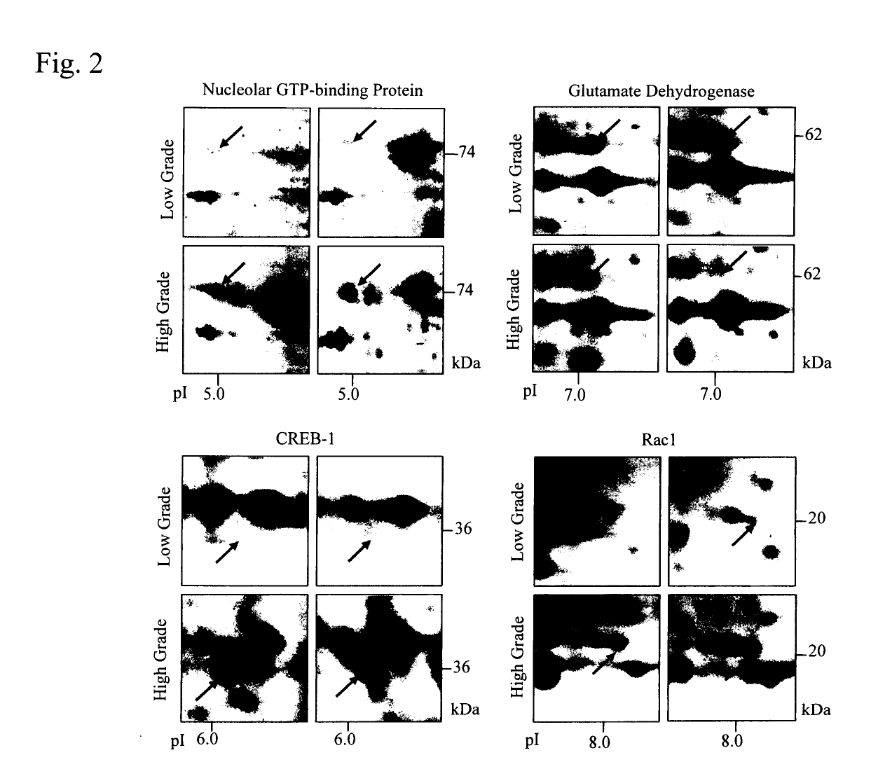

The protein expression was comprehensively analyzed in 75 glioma samples using two-dimensional electrophoresis technique of immobilized pH gradient method (FIG. 1). About 400 protein spots in each gel were identified using conservative spot selection software algorithm based on the constant spot intensity. Many protein spots were differently expressed for each of the samples (FIG. 2). The composite reference gel including 10 normal brain tissues included 631 protein spots in all. Matching of 64±6.2% was observed in average for astrocytoma by comparing the reference gel and gels from each sample of glioma, and matching of 47±4.9% (p <0.005) was observed in average for GM. 181 protein spots (28.7%) among the spots in the reference gel did not match with any spot in the other gel derived from glioma tissue. These proteins may be specifically translated and may function in normal brain. The rest of the proteins which did not match appeared only in one gel and therefore it is considered that tumor de-regulated proteins are extremely sample-specific and that they are proteolysis fragments and proteins modified after translation. There were also some protein spots which were possible to be identified by positive gel matching with protein expression patterns from some existing two-dimensional gel databases.

(2-2) Cluster Analysis of Proteome Profiling Pattern of Human Glioma

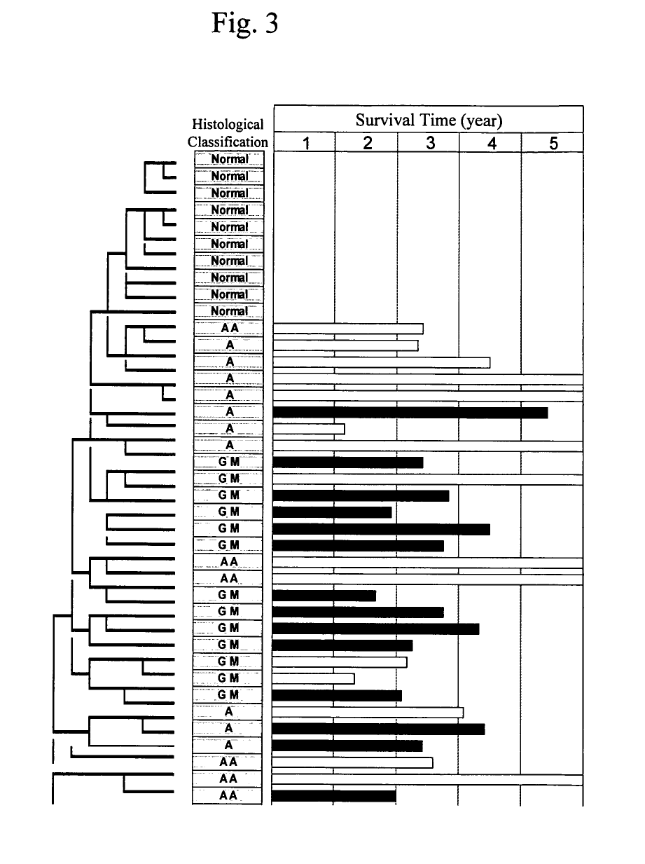

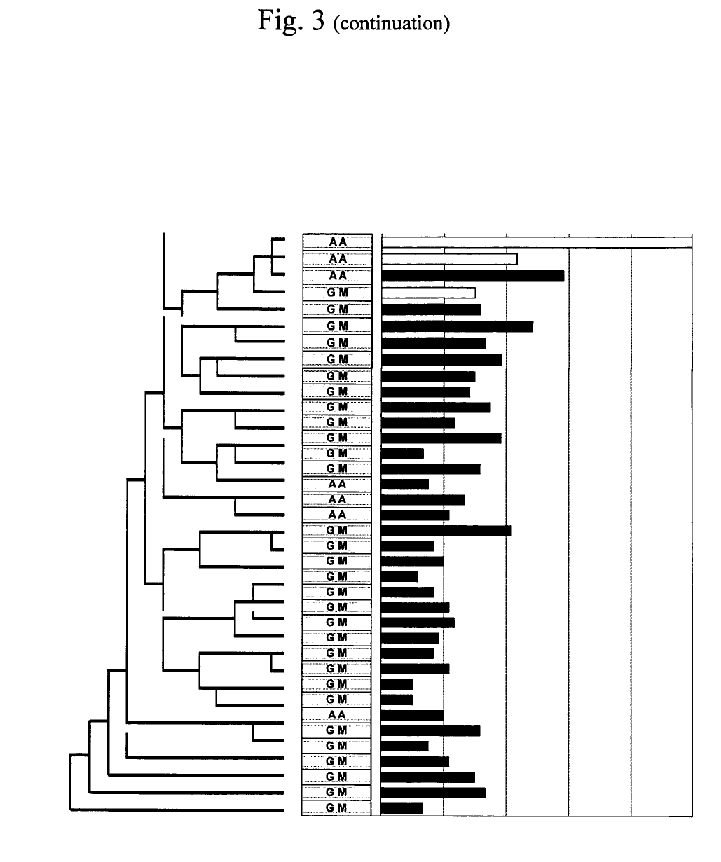

Hierarchical clustering of 75 tumor samples and 10 normal brain samples was performed based on the expression of the protein spots and a proteome profiling pattern in human glioma associated with various biochemical characteristics were identified. A phylogenetic tree on the sample axis is shown in FIG. 3. Samples obtained from normal brain tissues were completely classified into a single tissue specific branch, and appropriateness of the proteome analysis and clinical importance were demonstrated. Seven samples of astrocytoma gathered in the region near to that of the normal brain tissue, and the patient showed a good clinical course without recurrence. On the other hand, two people of the three remaining patients having astrocytoma and gathering away from the normal brain tissue had recurrence after the operation in 4 years or less. Most of GM having comparatively long survival time over two years were classified into a region near to that of low grade astrocytoma (FIG. 3).

(2-3) Identification of Proteins Associated with Progress of Malignancy in Glioma

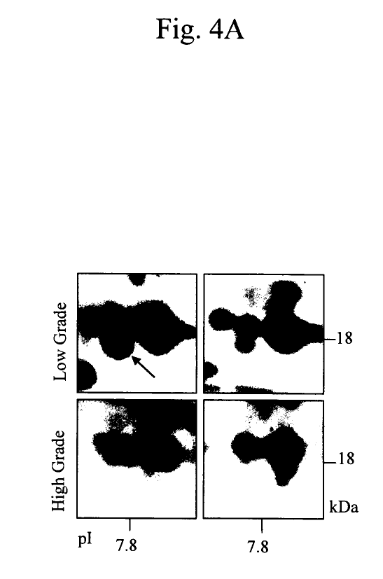

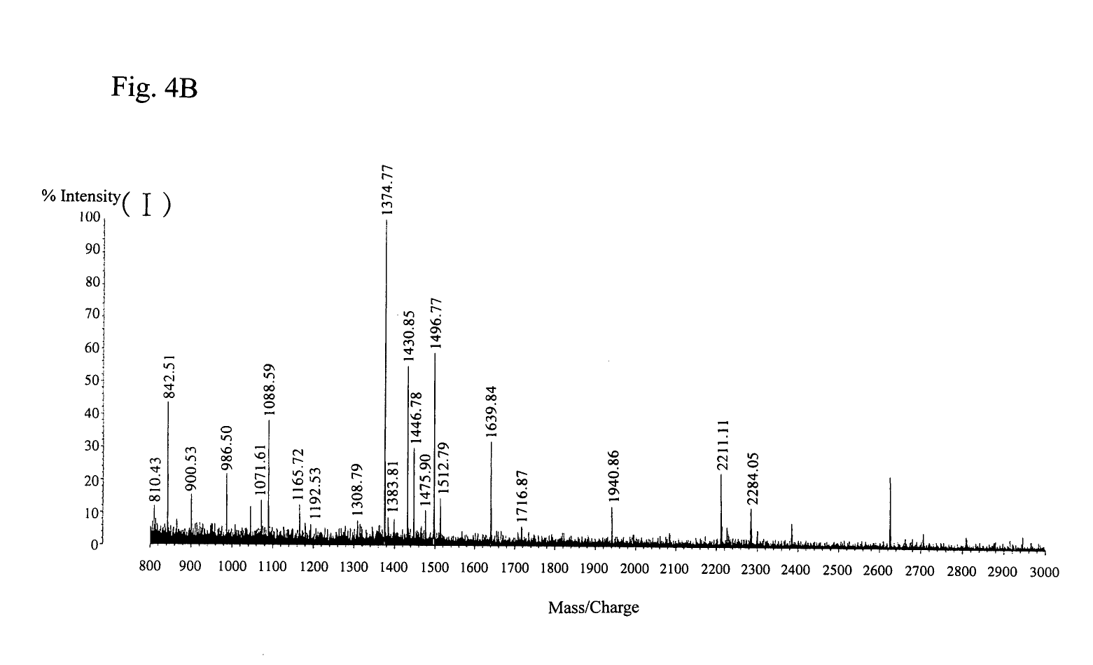

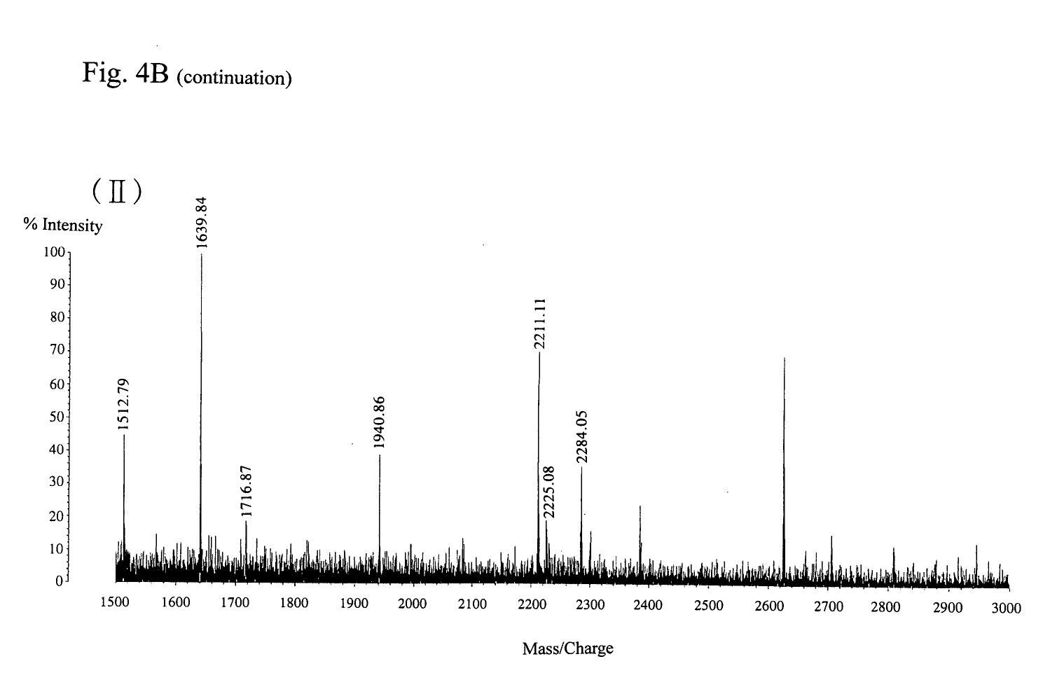

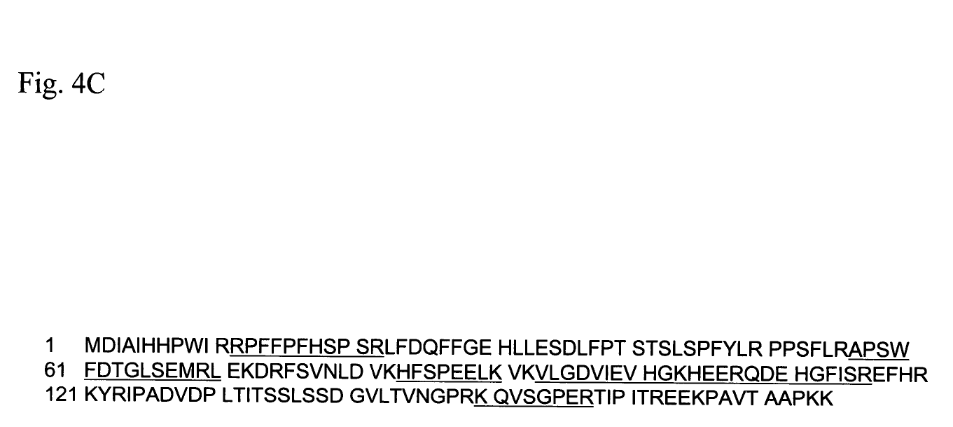

Identification analysis was carried out based on expression of protein spots in order to identify the proteins correlating to the histologic grade. The selected protein spots were excised and analyzed by MALDI-TOF MS assay (FIG. 4). 25 known proteins and 12 unknown proteins were extracted by the analysis. The 25 known proteins above were classified into signal transduction associated proteins, molecular chaperon, transcription and translation regulators, cell cycle mediated protein, extracellular matrix associated protein and cell adhesion molecule based on the known or supposed biological properties. 8 signal transduction proteins were upregulated in the high grade tumors, and 5 of these 8 proteins were classified into low molecular weight G protein superfamily. There have been no reports showing that RalA, Rab3B and nucleus GTP-binding protein, which have been recognized as a new G protein family in late years among the proteins identified here, were related to malignant transformation of glioma. The upregulated Rho family (RhoA and Racl) may cause inhibition of transfer of actin filament mediated tumor cells and apoptosis, partially through a group of proteins, ezrin/radixin/moesin (ERM) group. MAPK and that PKC have been also identified as proteins upregulated in high grade tumors. Annexin family, classified into phospholipid bond and Ca bond membrane associated enzymes, was frequently upregulated in high grade glioma. 4 molecular chaperons have shown correlation with histologic grade. 3 of these 4 proteins were significantly upregulated in low grade tumors. In addition, upregulation or downregulation of three metabolism enzyme, phosphoglycerate mutase, glutamate dehydrogenase and phosphopyruvate hydratase have shown significant correlation with histologic grade.

### Example 2

Protein markers associated with drug sensitivity and drug resistance of brain tumor were identified.

A drug sensitivity test using flow cytometry followed a method described in a reference (Iwadate,Y., et al., Int J Cancer 69:236-240, 1996; Iwadate, Y., et al., Int J Mol Med 10: 187-192, 2002). That is, a tumor cell suspension was incubated together with 30 kinds of different anti-cancer agents for 8 hours and subsequently further cultured in a fresh culture medium which did not include a drug. These drugs were classified into five following categories by pharmacological mechanism.
Alkylating agents:
4-hydroperoxy-cyclophosphamide (CPM)(1 μg/ml), 4-hydroperoxy-ifosfamide (IFOS) (I μg/ml), melphalan (MPL) (0.5 μg/ml), carboquone (CQ) (0.1 μg/ml), nimustine (ACNU) (2 μg/ml), ranimustine (MCNU) (2 μg/ml), cisplatinum (CDDP) (0.5 μg/ml) and carboplatin (JM-8) (4 μg/ml)
Antimetabolites:
Methotrexate (MTX) (3 μg/ml), 5-fluoro uracil (5-FU) (10 μg/ml), thioinosine (6-MRP) (3μg/ml) and cytosine arabinoside (CA) (4 μg/ml)
Antibiotics:
Mitomycin C (MMC) (0.2 μg/ml), bleomycin (BLM) (1 μg/ml), peplomycin (PEP) (0.5 μg/ml), chromomycin A3 (TM) (0.01 μg/ml) and neocarsinostatin (NCS) (0.15 μg/ml)
Topoisomerase inhibitors:
Actinomycin D (ACD) (0.01 μg/ml), aclarubicin (ACR) (0.6 μg/ml), adriamycin (ADM) (0.3 μg/ml), daunomycin (DM) (0.6 μg/ml), pirarubicin (THP) (0.3 μg/ml), epirubicin (4′-EPI) (0.4 μg/ml), mitoxantrone (MIT) (0.06 μg/ml), etoposide (VP-16) (3 μg/ml) and camptothecine (CPT-11) (3 μg/ml)
Microtubule Inhibitors:
Vincristine (VCR) (0.1 μg/ml), vinblastin (VLB) (0.1 μg/ml), vindesin (VDS) (0.1 μg/ml) and paclitaxel (TAX) (0.6 μg/ml)

The in vitro concentrations of these drugs were set to about 1/10 of the highest blood concentration when administered at a dose clinically recommended, and differences in the reaction by dose were evaluated for 10 selected drugs (CPM, ACNU, CDDP, PEP, ADM, VP-16, CA, TAX, VCR). The cultured samples of these were mixed in a phosphate buffered saline (pH 7.2)/0.1% Triton X-100/0.1 mg/ml RNase/0.01% sodium azide for 15 minutes and subsequently mixed with 50 g/ml of propidium iodide (Sigma,St.Louis, Mo.) for 5 minutes. The isolated nuclei were analyzed with flow cytometer (FACScan:Becton Dickinson, Mountain view, Calif.). The efficacy of a drug was judged by shifting the nucleus of apoptosis to a low diploid (sub-G1) region and comparing reverse decrease of the peak of the thus incorporated diploid with decrease of untreated control cells. Cells were fixed with 70% ethanol and subjected to Giemsa's staining on a slide glass to morphologically confirm the drug induced cell death (Iwadate, Y., et al., Int J Mol Med 10:187-192, 2002). Decomposition and condensation of chromatin were judged as a marker of apoptosis and quantitatively evaluated by Fisher's exact test. This morphologic inspection revealed that the average ratio of tumor cells in the preparation unalterably exceeded 95%. The integrity of DNA evaluated by FCM correlated well to the morphologic change by apoptosis. In order to avoid false-negative results, when either one of the assays was judged to be positive, the drug was eventually determined to be effective in vitro.

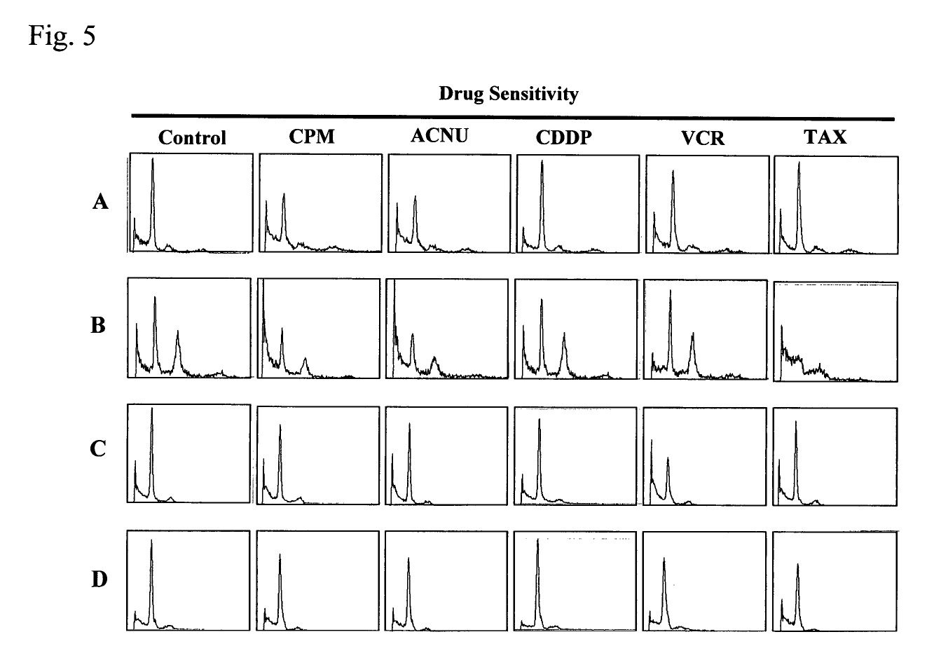

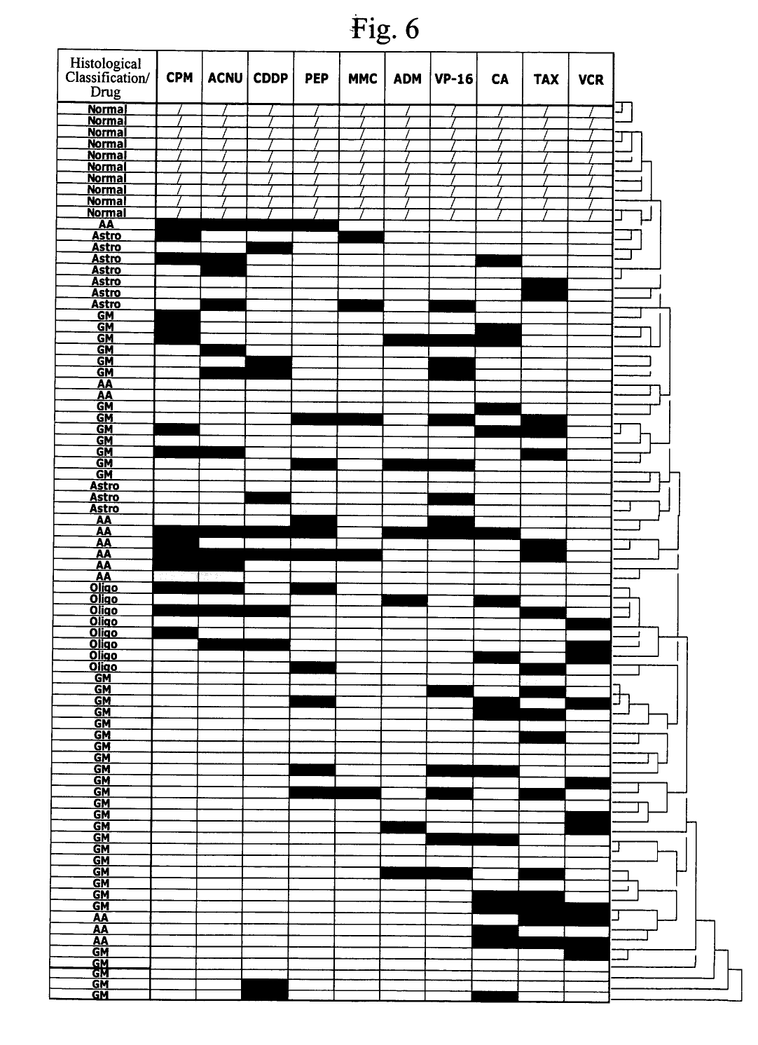

Correlation between the degree of drug sensitivity obtained by the above-mentioned method and proteome profiling patterns was examined. The drug sensitivity showed variability case by case (FIG. 5). The drug sensitivity showed characteristic distribution (FIG. 6) in the phylogenetic tree in the sample axis obtained in the same way as in Example 1 (2). That is, an alkylating agent has a high efficacy for the tumor which gathered to the neighborhood to the normal brain tissue, but it was not effective at all for the tumor which gathered in a remote space from the normal brain tissue. A tumor having sensitivity to ACNU, one of the alkylating agent, was generally resistant to VCR, one of the microtubule inhibitor. Most of the tumors resistant to all the drugs gathered in a remote space from the normal brain tissue.

The tumors resistant to all the drugs were considered to be tumors which lost a cell death mechanism by apoptosis, and therefore, identification analysis was conducted based on presence/absence of effective drugs as in the same way as in Example 1 (2). As a result, 30 known proteins and 12 unknown proteins were extracted.

### INDUSTRIAL APPLICABILITY

As described in detail heretofor, the invention of this application enables to precisely diagnose the grade of malignancy and/or drug sensitivity of brain tumor, and enables to select more effective therapies.

1. A brain tumor marker, characterized in that the brain tumor marker is at least one protein expressed in a brain tumor tissue and selected from the group of proteins having the amino acid sequences represented by SEQ ID NOS: 1 to 16, respectively, wherein the protein is expressed in at least one tissue selected from the group of the tissues of a highly malignant brain tumor, a low malignant brain tumor, a drug-sensitive brain tumor, and a drug-resistant brain tumor.

2. A highly malignant brain tumor marker, wherein the brain tumor marker is at least one protein selected from the group of proteins expressed in a highly malignant brain tumor tissue, wherein the protein has the amino acid sequence represented by SEQ ID NO: 1, 4, 5, 6 or 7.

3. A low malignant brain tumor marker, wherein the brain tumor marker is at least one protein selected from the group of proteins expressed in a low malignant brain tumor tissue, wherein the protein has the amino acid sequence represented by SEQ ID NO: 2 or 3.

4. A drug-sensitive brain tumor marker, wherein the brain tumor marker is at least one protein selected from the group of proteins expressed in a drug-sensitive brain tumor tissue, wherein the protein has the amino acid sequence represented by SEQ ID NO: 5,8,11,13, 14, 15 or 16.

5. A drug-resistant brain tumor marker, wherein the brain tumor marker is at least one protein selected from the group of proteins expressed in a drug-resistant brain tumor tissue, wherein the protein has the amino acid sequence represented by SEQ ID NO: 1, 2, 9, 10, 12 or 16.

6. An antibody which binds to a protein selected from the group of proteins having the amino acid sequences represented by SEQ ID NOS: 1 to 16, respectively.

7. A diagnostic kit, characterized in that the diagnostic kit comprises at least one of the antibody according to claim 6 and/or a labeled antibody derived therefrom.

8. A polynucleotide encoding the highly malignant brain tumor marker according to claim 2.

9. A polynucleotide encoding the low malignant brain tumor marker according to claim 3.

10. A polynucleotide encoding the drug-sensitive brain tumor marker according to claim 4.

11. A polynucleotide encoding the drug-resistant brain tumor marker according to claim 5.

12. A DNA fragment consisting of a partial contiguous sequence of any one of SEQ ID NOS: 17 to 32.

13. A DNA microarray comprising the polynucleotide according to claim 8 and/or the DNA fragment consisting of a partial contiguous sequence of any one of SEQ ID NOS: 17 to 32.

14. A set of primers for amplifying by PCR any one of the nucleotide sequences of SEQ ID NOS: 17 to 32.

15. A method of diagnosing a highly malignant brain tumor, characterized in that a biological sample from a subject is assayed for the expression level of the polynucleotide according to claim 8, wherein the subject is judged to be a patient with a highly malignant brain tumor if the subject has a higher expression level of at least one said polynucleotide than a healthy person.

16. A method of diagnosing a low malignant brain tumor, characterized in that a biological sample from a subject is assayed for the expression level of the polynucleotide according to claim 9, wherein the subject is judged to be a patient with a low malignant brain tumor if the subject has a higher expression level of at least one said polynucleotide than a healthy person.

17. A method of diagnosing a drug-sensitive brain tumor, characterized in that a biological sample from a subject is assayed for the expression level of the polynucleotide according to claim 10, wherein the subject is judged to be a patient with a drug-sensitive brain tumor if the subject has a higher expression level of at least one said polynucleotide than a healthy person.

18. A method of diagnosing a drug-resistant brain tumor, characterized in that a biological sample from a subject is assayed for the expression level of the polynucleotide according to claim 11, wherein the subject is judged to be a patient with a drug-resistant brain tumor if the subject has a higher existence level of at least one said polynucleotide than a healthy person.

19. A method of diagnosing a brain tumor, characterized in that said method combines the respective methods according to claim 15, wherein a brain tumor which a subject suffers from is judged to be one selected from the group of
(a) highly malignant and drug-sensitive;
(b) highly malignant and drug-resistant;
(c) low malignant and drug-sensitive; and
(d) low malignant and drug-resistant.
